(19) United States
(12) Patent Application Publication
(10) Pub. No.: US 2022/0076799 A1
(43) Pub. Date: Mar. 10, 2022

(54) MACHINE LEARNING TECHNIQUES FOR DETERMINING THERAPEUTIC AGENT DOSAGES

(71) Applicant: President and Fellows of Harvard College, Cambridge, MA (US)

(72) Inventor: Dalit Engelhardt, Cambridge, MA (US)

(21) Appl. No.: 17/420,814

(22) PCT Filed: Jan. 7, 2020
(86) PCT No.: PCT/US2020/012543

§ 371 Date: Jul. 6, 2021

Related U.S. Application Data
(60) Provisional application No. 62/789,238, filed on Jan. 7, 2019.
(60) Provisional application No. 62/823,203, filed on Mar. 25, 2019.

(51) Int. Cl. G16H 20/13; G16H 50/20; G16B 20/00; G06N 3/04; G06N 3/08

(52) U.S. Cl. CPC G16H 20/13; G16H 50/20; G06N 3/08; G06N 3/0454; G16B 20/00

(57) ABSTRACT

Techniques for determining a dosage plan for administering at least one therapeutic agent to a subject. The techniques include using at least one computer hardware processor to perform: accessing information specifying a plurality of cell population concentrations for a respective plurality of cell populations in a biological sample from a subject; and determining the dosage plan using a trained statistical model and the plurality of cell population concentrations, the dosage plan including one or more concentrations of the at least one therapeutic agent to be administered to the subject at one or more respective different times. The trained statistical model may be trained using an actor-critic reinforcement learning technique and a model of cell evolution. The trained statistical model may include a deep neural network.

### CROSS-REFERENCE TO RELATED APPLICATIONS

The present application claims the benefit of priority under 35 U.S.C. § 119(e) to U.S. Provisional Application Ser. No. 62/789,238, titled “MACHINE LEARNING TECHNIQUES FOR DETERMINING THERAPEUTIC AGENT DOSAGES”, filed on Jan. 7, 2019 (Attorney Docket No.: H0776.70112US00) and to U.S. Provisional Application Ser. No. 62/823,203, titled “MACHINE LEARNING TECHNIQUES FOR DETERMINING THERAPEUTIC AGENT DOSAGES”, filed on Mar. 25, 2019 (Attorney Docket No.: H0776.70112US01), each of which is incorporated by reference in its entirety.

### FEDERALLY SPONSORED RESEARCH

This invention was made with government support under GM124044 awarded by the National Institutes of Health. The government has certain rights in the invention.

### BACKGROUND

The development of drug resistance in response to treatment is a major cause of treatment failure across a variety of diseases, patient populations, and administered drugs. Although preventing and/or overcoming drug resistance is critical to achieving successful treatment outcomes, developing dosage plans for treatments that effectively prevent and/or overcome drug resistance has remained challenging.

### SUMMARY

Some embodiments provide a computer-implemented method for determining a dosage plan for administering at least one therapeutic agent to a subject, wherein the method comprises: using at least one computer hardware processor to perform: accessing information specifying a plurality of cell population concentrations for a respective plurality of cell populations in a biological sample from a subject; and determining the dosage plan using a trained statistical model and the plurality of cell population concentrations, the dosage plan including one or more concentrations of the at least one therapeutic agent to be administered to the subject at one or more respective different times.

In some embodiments, determining the dosage plan comprises: determining the one or more concentrations of the at least one therapeutic agent; and determining the one or more respective different times for administering the one or more concentrations of the at least one therapeutic agent;

In some embodiments, determining the dosage plan comprises: accessing information specifying the one or more respective different times; and determining the one or more concentrations of the at least one therapeutic agent for the one or more respective different times.

In some embodiments, determining the dosage plan comprises: providing the plurality of population concentrations as input to the trained statistical model; and determining the dosage plan using the output of the trained statistical model.

In some embodiments, each one of the plurality of cell populations has respective dose-response characteristics different from dose-response characteristics of other ones of the plurality of cell populations.

In some embodiments, the at least one therapeutic agent includes a first therapeutic agent, wherein the plurality of cell populations includes a first cell population associated with first dose-response characteristics for the first therapeutic agent and a second cell population associated with second dose-response characteristics for the first therapeutic agent, and wherein the first dose-response characteristics are different from the second dose-response characteristics.

In some embodiments, a measure of difference between the first dose-response characteristics and the second dose-response characteristics is above a threshold.

In some embodiments, the first dose-response characteristics comprise first parameters for a first dose-response curve and the second dose response characteristics comprise second parameters for a second dose-response curve, wherein the first parameters are different from the second parameters.

In some embodiments, the trained statistical model includes a neural network model.

In some embodiments, the neural network model includes a deep neural network model.

In some embodiments, the deep neural network model includes one or more convolutional layers.

In some embodiments, the deep neural network model includes one or more fully connected layers.

In some embodiments, the trained statistical model is trained using a reinforcement learning technique.

In some embodiments, the trained statistical model is trained using an actor-critic reinforcement learning technique.

In some embodiments, the trained statistical model includes a trained actor network trained using an actor-critic reinforcement learning algorithm.

In some embodiments, the trained statistical model is trained using a deep deterministic policy gradient algorithm.

In some embodiments, the trained statistical model is trained using training data generated using at least one model of cell population evolution.

In some embodiments, the training data is generated using stochastic simulations of the at least one model of cell population evolution.

In some embodiments, the at least one model of cell population evolution includes a set of differential equations representing a time evolution of the plurality of cell population concentrations for the respective plurality of cell populations.

In some embodiments, the at least one therapeutic agent includes a first therapeutic agent, and wherein the set of differential equations depends on dose-response characteristics for the first therapeutic agent.

In some embodiments, the at least one model of cell population evolution models concentration changes among cell populations in the plurality of cell populations.

In some embodiments, the at least one model of cell population evolution models birth of a new cell population.

In some embodiments, the at least one model of cell population evolution models death of a cell population in the plurality of cell populations.

In some embodiments, the at least one therapeutic agent is a small molecule, a protein, a nucleic acid, gene therapy, a drug approved by regulatory approval agency, a biological product approved by a regulatory approval agency, or some combination thereof.

In some embodiments, the method further comprises administering the at least one therapeutic agent to the subject in accordance with the dosage plan.

Some embodiments provide at least one non-transitory computer-readable storage medium storing processor-executable instructions that, when executed by a computer hardware processor, cause the computer hardware processor to perform a method for determining a dosage plan for administering at least one therapeutic agent to a subject, the method comprising: accessing information specifying a plurality of cell population concentrations for a respective plurality of cell populations in a cell environment from a subject; and determining the dosage plan using a trained statistical model and the plurality of cell population concentrations, the dosage plan including one or more concentrations of the at least one therapeutic agent to be administered to the subject at one or more respective different times.

Some embodiments provide a system, comprising: at least one computer hardware processor; and at least one non-transitory computer-readable storage medium storing processor-executable instructions that, when executed by the computer hardware processor, cause the computer hardware processor to perform a method for determining a dosage plan for administering at least one therapeutic agent to a subject, the method comprising: accessing information specifying a plurality of cell population concentrations for a respective plurality of cell populations in a cell environment from a subject; and determining the dosage plan using a trained statistical model and the plurality of cell population concentrations, the dosage plan including one or more concentrations of the at least one therapeutic agent to be administered to the subject at one or more respective different times.

Some embodiments provide a computer-implemented method for training a statistical model to determine a dosage plan for administering at least one therapeutic agent to a subject, the method comprising: using at least one computer hardware processor to perform: generating training data for training the statistical model at least in part by using information specifying an initial plurality of cell population concentrations for a respective plurality of cell populations, and at least one model of cell population evolution; training the statistical model using the training data to obtain a trained statistical model; and storing the trained statistical model.

In some embodiments, the method further comprises accessing information specifying, for the subject, a plurality of cell population concentrations for the respective plurality of cell populations; and determining, using the trained statistical model and the plurality of cell population concentrations, the dosage plan for administering the at least one therapeutic agent to the subject, wherein the dosage plan includes one or more concentrations of the at least one therapeutic agent to be administered to the subject at one or more respective different times.

In some embodiments, each one of the plurality of cell populations has respective dose response characteristics different from dose-response characteristics of other ones of the plurality of cell populations.

In some embodiments, training the statistical model comprises using a reinforcement learning technique.

In some embodiments, training the statistical model comprises using an actor-critic reinforcement learning technique.

In some embodiments, training the statistical model comprises using a deep deterministic policy gradient algorithm.

In some embodiments, the training data is generated using stochastic simulations of the at least one model of cell population evolution.

In some embodiments, the at least one model of cell population evolution includes a set of differential equations representing a time evolution of the initial plurality of cell population concentrations for the respective plurality of cell populations.

In some embodiments, the at least one therapeutic agent includes a first therapeutic agent, and wherein the set of differential equations depends on dose-response characteristics for the first therapeutic agent.

Some embodiments provide at least one non-transitory computer-readable storage medium storing processor-executable instructions that, when executed by a computer hardware processor, cause the computer hardware processor to perform a method for training a statistical model to determine a dosage plan for administering at least one therapeutic agent to a subject, the method comprising: generating training data for training the statistical model at least in part by using information specifying an initial plurality of cell population concentrations for a respective plurality of cell populations, and at least one model of cell population evolution; training the statistical model using the training data to obtain a trained statistical model; and storing the trained statistical model.

Some embodiments provide a system, comprising: at least one computer hardware processor; and at least one non-transitory computer-readable storage medium storing processor-executable instructions that, when executed by the computer hardware processor, cause the computer hardware processor to perform a method for training a statistical model to determine a dosage plan for administering at least one therapeutic agent to a subject, the method comprising: generating training data for training the statistical model at least in part by using information specifying an initial plurality of cell population concentrations for a respective plurality of cell populations, and at least one model of cell population evolution; training the statistical model using the training data to obtain a trained statistical model; and storing the trained statistical model.

Some embodiments provide a method for performing controlled evolution of cells within a cell environment, the method comprising: (A) accessing information specifying a plurality of cell population concentrations for a respective plurality of cell populations in the cell environment; (B) using a trained statistical model and the plurality of cell population concentrations to determine one or more concentrations of at least one agent to be administered to the cell environment; and (C) administering the at least one agent to the cell environment in the determined one or more concentrations.

In some embodiments, the acts (A), (B), and (C) are performed repeatedly.

In some embodiments, the method is performed by automated lab machinery comprising at least one computer hardware processor.

In some embodiments, the trained statistical model is trained using training data generated using at least one model of cell population evolution.

In some embodiments, the training data is generated using stochastic simulations of the at least one model of cell population evolution.

In some embodiments, the at least one model of cell population evolution includes a set of differential equations representing a time evolution of the plurality of cell population concentrations for the respective plurality of cell populations.

In some embodiments, the at least one agent includes a first agent and the set of differential equations depends on dose-response characteristics for the first agent.

In some embodiments, the trained statistical model includes a neural network model.

Some embodiments provide at least one non-transitory computer-readable storage medium storing processor-executable instructions that, when executed by a computer hardware processor, cause the computer hardware processor to perform a method for performing controlled evolution of cells within a cell environment, the method comprising: (A) accessing information specifying a plurality of cell population concentrations for a respective plurality of cell populations in the cell environment; (B) using a trained statistical model and the plurality of cell population concentrations to determine one or more concentrations of at least one agent to be administered to the cell environment; and (C) administering the at least one agent to the cell environment in the determined one or more concentrations.

Some embodiments provide a system, comprising: at least one computer hardware processor; and at least one non-transitory computer-readable storage medium storing processor-executable instructions that, when executed by the computer hardware processor, cause the computer hardware processor to perform a method for performing controlled evolution of cells within a cell environment, the method comprising: (A) accessing information specifying a plurality of cell population concentrations for a respective plurality of cell populations in the cell environment; (B) using a trained statistical model and the plurality of cell population concentrations to determine one or more concentrations of at least one agent to be administered to the cell environment; and (C) administering the at least one agent to the cell environment in the determined one or more concentrations.

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a diagram of an illustrative clinical setting in which some embodiments of the technology described herein may be used;

FIG. 1B is a diagram of illustrative treatments being applied to cell populations, in accordance with some embodiments of the technology described herein;

FIG. 2 is a flowchart of an illustrative method for determining a dosage plan for administering at least one therapeutic agent to a subject, in accordance with some embodiments of the technology described herein;

FIG. 3 is a flowchart of an illustrative method for training a statistical model to determine a dosage plan for administering at least one therapeutic agent to a subject, in accordance with some embodiments of the technology described herein;

FIG. 4 is a diagram illustrating developing a model of cell population evolution, in accordance with some embodiments of the technology described herein;

FIG. 5 is a chart showing exemplary dose-response characteristics for some cell populations, in accordance with some embodiments of the technology described herein;

FIG. 6 is a chart showing sample cell population evolution simulations, in accordance with some embodiments of the technology described herein;

FIG. 7 is a diagram illustrating aspects of training a statistical model using reinforcement learning, in accordance with some embodiments of the technology described herein;

FIG. 8A-8B is a flowchart showing an illustrative process 800 for training a statistical model for determining a dosage plan, in accordance with some embodiments of the technology described herein;

FIG. 9A-9B is a flowchart showing an illustrative implementation of act 850 of process 800 for training a statistical model for determining a dosage plan, in accordance with some embodiments of the technology described herein;

FIG. 10A-10C are diagrams showing pseudocode for an example implementation of a training process for training a statistical model for determining a dosage plan, in accordance with some embodiments of the technology described herein;

FIG. 11 is a flowchart of an illustrative process 1100 for performing controlled evolution of cells within a biological sample, in accordance with some embodiments of the technology described herein;

FIG. 12A-12E are charts showing exemplary results of a statistical model trained using the training process of FIG. 8A-8B in accordance with some embodiments of the technology described herein;

FIG. 13A-13D are charts showing exemplary training loss and reward of a statistical model trained using the training process of FIG. 8A-8B, in accordance with some embodiments of the technology described herein; and

FIG. 14 is a block diagram of an illustrative computer system that may be used in implementing some embodiments of the technology described herein.

### DETAILED DESCRIPTION

The inventor(s) have developed machine learning techniques for determining a dosage plan for administering one or more therapeutic agents to a patient (or for applying the therapeutic agent(s) in vitro to a biological sample) that reduces or eliminates the possibility that the patient (or biological sample) develops resistance to the therapeutic agent(s). In some embodiments, the dosage plan may be determined by a trained statistical model (e.g., a deep neural network) based on information specifying concentrations of cell populations in the patient (or biological sample). In some embodiments, the trained statistical model may be trained using a model of cell population evolution and reinforcement learning techniques, for example, actor-critic reinforcement learning techniques.

The inventor(s) have appreciated that the emergence of resistance during treatment may be closely related to the development of high-resistance cell populations that can arise randomly and at highly variable rates within a cell environment. The emergence and potential rise to dominance of resistant cell populations are stochastic processes driven by the dynamic and complex interaction between the influences and interplay of natural selection, demographic stochasticity, and environmental fluctuations. In some cases, resistance to a particular therapeutic agent used in treatment can be present prior to treatment, but in others it may emerge during treatment through a diverse range of mechanisms. Additionally, drug resistance may evolve dynamically and non-uniformly: for example, variability among cells can lead to faster adaptation to environmental pressures and thus promote the rise of resistant cell populations even among cell populations initially susceptible to the therapeutic agent. Moreover, treatments having improper dosage plans can exert a selective evolutionary pressure that may lead to the elimination of susceptible cell populations but leave more resistant cell populations largely unaffected and able to proliferate due to reduced competition with susceptible cell populations. As a result, determining dosage plans for treatments in cell environments prone to resistance evolution has remained challenging.

The inventor(s) have recognized that control theory and/or reinforcement learning techniques may be applied to the problem of determining dosage plans in cell environments prone to the evolution of drug-resistive cell populations. This is notable because although the application of reinforcement learning techniques to a variety of control tasks has seen significant advances in recent years, conventional reinforcement learning techniques have not been applied to highly stochastic and realistic environments, such as cell environments. In part, this is because conventional reinforcement learning techniques are not sufficiently adaptable and robust to handle the unexpected feedback that may arise frequently in cell environments prone to resistance evolution, where theoretical guarantees may not be available. As such, both conventional control methods and simple reinforcement learning techniques (e.g., tabular learning) are inappropriate for realistic cell evolution scenarios. Significant and variable stochasticity, nonlinearity in the dynamics, a potentially high-dimensional space of biological units (e.g., cell populations), and parameter uncertainty all pose significant challenges to the application of traditional control methods to such complex biological systems.

This is especially problematic in real-world (e.g., clinical or in vitro) applications, where, as the inventors have recognized, a verified ability to learn control policies capable of efficient generalization across parameter values not seen during training is crucial for safe implementation. The inventors have also recognized the importance of the ability to discover, despite system stochasticity and random events, high-preference low-cost policies applicable when such perturbations do not occur. However, due to the lack of adaptability and robustness, conventional techniques have failed to effectively address these concerns.

The inventor(s) have appreciated that another impediment to applying conventional techniques for determining dosage plans for treatments in cell environments prone to resistance evolution is that such techniques must balance the need for efficiently targeting all cell populations that may either exist at the onset of treatment or that may spontaneously emerge during treatment, while maintaining sufficiently low dosing for toxicity minimization. For example, a resistant cell population may be suppressed more efficiently with a higher-toxicity therapeutic agent, but increased dosages of the higher-toxicity therapeutic agent may result in negative treatment outcomes for the subject due to the heightened toxicity. Despite the importance of this trade-off in determining effective and safe dosage plans for treatments, effectively managing the balance between these factors has remained challenging with conventional techniques. Indeed, conventional techniques have failed to effectively account for these factors.

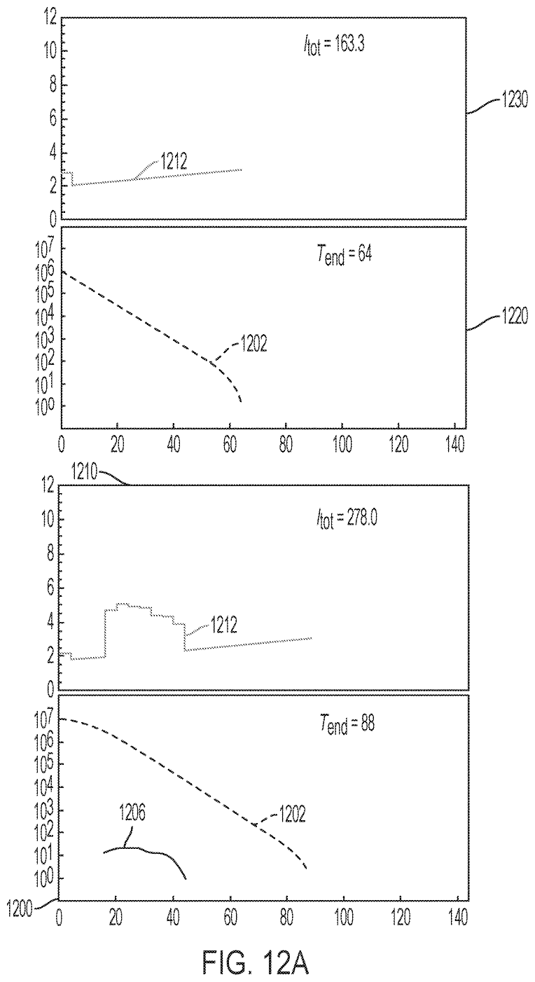

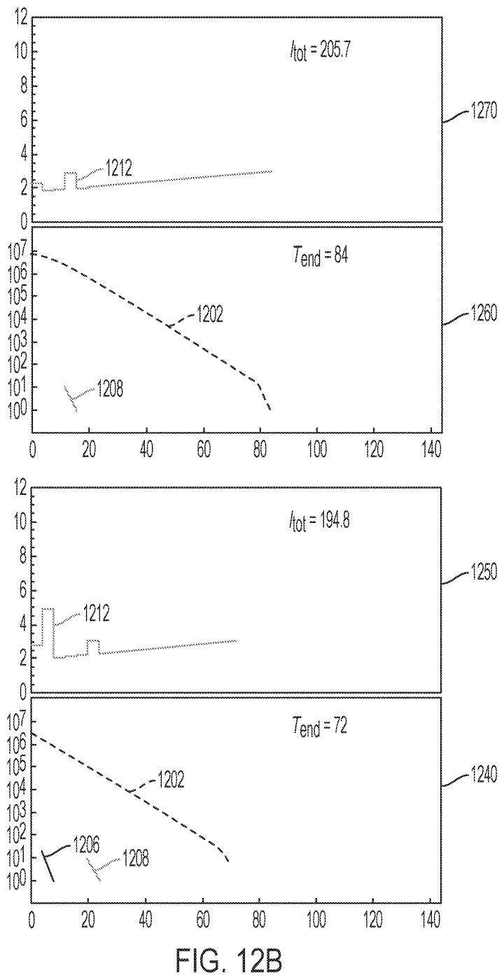

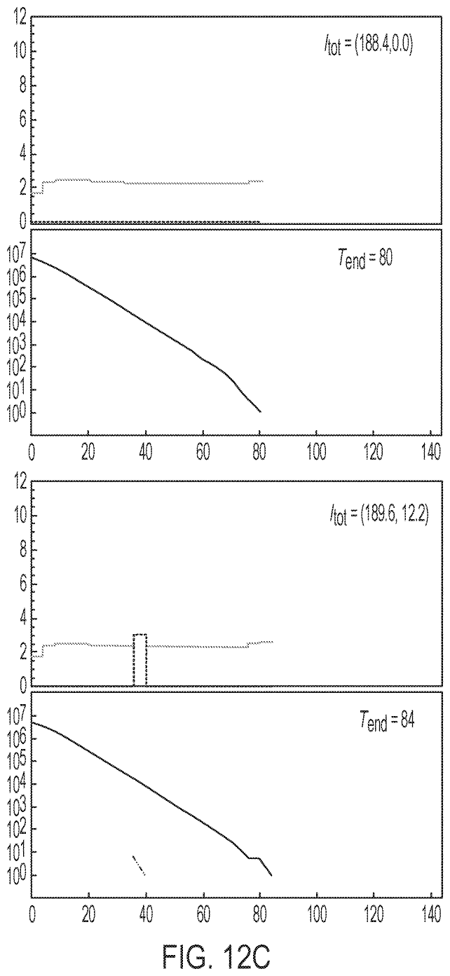

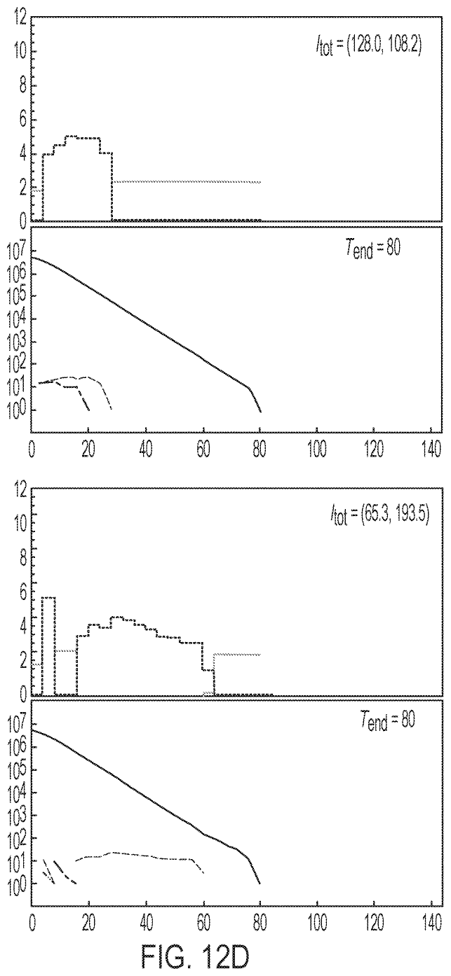

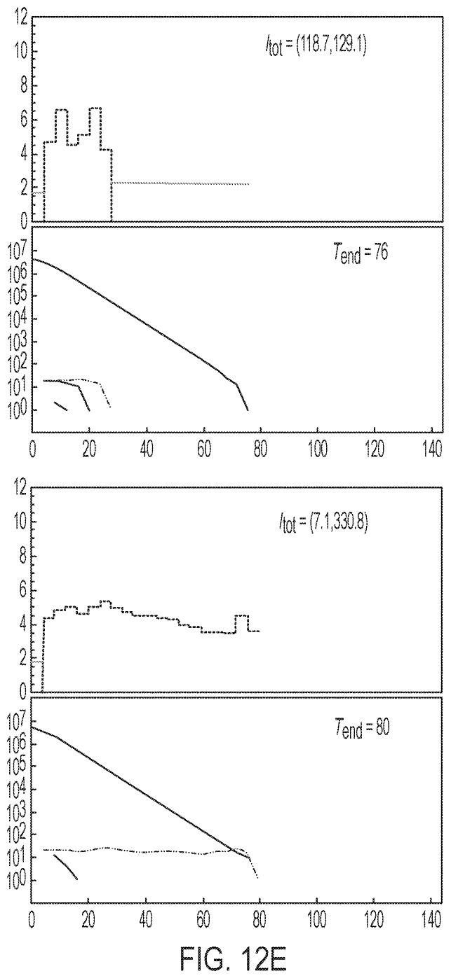

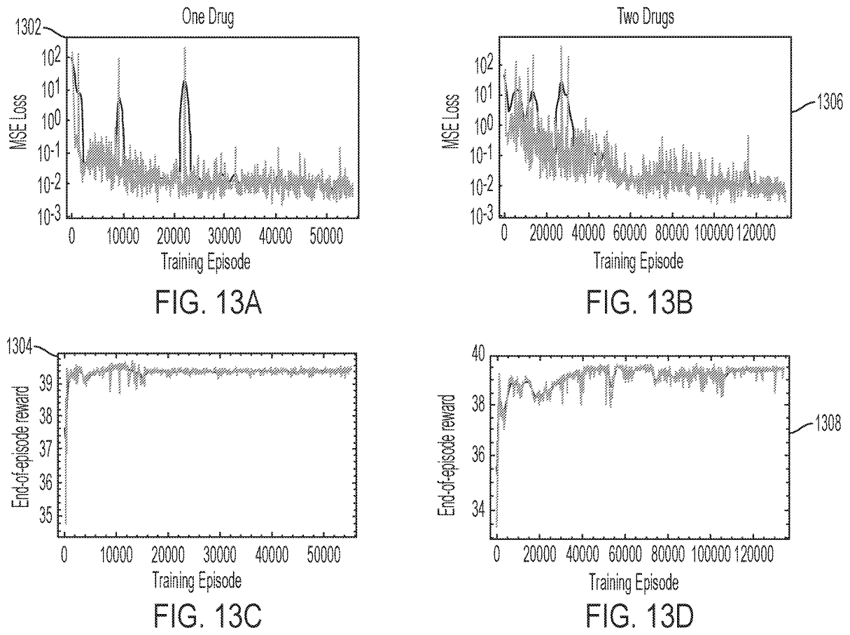

As such, the inventor(s) have recognized and appreciated the need for robust techniques for determining dosage plans that account for the complex, stochastic nature of cell population dynamics, and adaptively target cell populations having a variety of drug susceptibility and resistance characteristics. Accordingly, the inventor(s) have developed techniques for using a neural network model trained using actor-critic reinforcement learning techniques to determine improved dosage plans, based on information about cell populations of the subject being treated. As described herein, these techniques serve to combat the emergence of drug resistance during treatment by sensitively adjusting dosages and/or switching the administered therapeutic agents in response to observed feedback from the cell environment, while employing minimal overall dosage for toxicity reduction. These techniques present a significant improvement over the prior art, as described herein, including with respect to FIGS. 12 and 13.

Accordingly, some embodiments provide for a computer-implemented method for determining a dosage plan for administering one or multiple therapeutic agents (e.g., drugs for treatment) to a subject (e.g., a patient being treated in a clinical setting, a mouse or other animal in an experimental setting, a biological sample, or any other cell environment). This method may involve: (1) accessing information specifying cell population concentrations for respective cell populations within a biological sample from the subject (e.g., from the patient, such as may be collected by the clinician in a clinical setting, or from any other suitable cell environment); and (2) determining the dosage plan using a trained statistical model (e.g., a neural network model, which may be trained using actor-critic reinforcement learning techniques, in some embodiments) and the cell population concentrations. The dosage plan may include concentrations of one or more therapeutic agents to be administered to the subject at different times (e.g., doses of one or more drugs that may be given to the patient at discrete time intervals, or administered continuously, such as in the case of intravenous administration).

In some embodiments, determining the dosage plan includes determining the different concentrations of the therapeutic agent(s) and the times at which the agent(s) are to be administered to the subject. Alternatively or additionally, determining the dosage plan may involve determining the different concentrations of the therapeutic agents, then accessing information specifying the different times at which it is to be administered (e.g., if the time intervals pre-established by constraints of drug administration, the preference of a clinician, etc.). In order to determine the dosage plan using the trained statistical model and the cell population concentrations, the cell population concentrations may be provided as input to the trained statistical model, and the dosage plan may be determined using the output of the trained statistical model.

In some embodiments, cell populations within the biological sample from the subject may have different dose-response characteristics for the same underlying therapeutic agent or agents. For example, cell populations in a biological sample of a patient may have varying degrees of susceptibility or resistance to particular drugs. For example, for a given therapeutic agent, one cell population may have certain dose-response characteristics for that therapeutic agent, whereas another cell population may have different dose-response characteristics for the same therapeutic agent. In some cases, the difference between first dose-response characteristics of a first cell population, and second dose response characteristics of a second cell population may be above a threshold. Alternatively or additionally the first dose-response characteristics may include first parameters for a first dose-response curve, and the second dose response characteristics may include second parameters for a second dose-response curve, such that the first parameters are different from the second parameters.

In some embodiments, the trained statistical model may include a neural network model, such as, for example, a deep neural network model. In some cases, the deep neural network model may include one or more convolutional layers. In some cases, it may include one or more fully connected layers. As described above, the trained statistical model may be trained using a reinforcement learning technique. More particularly, it may be trained using an actor-critic reinforcement learning technique. In some cases, the trained statistical model may include a trained actor network trained using an actor-critic reinforcement learning algorithm. For example, the trained statistical model may be trained using a deep deterministic policy gradient (DDPG) algorithm or any other suitable actor-critic reinforcement learning algorithm, as aspects of the technology described herein are not limited to training statistical models using the DDPG algorithm.

In some embodiments, the trained statistical model may be trained using training data generated using a model of cell population evolution. This training data may be generated using stochastic simulations of the model of cell population evolution. The model of cell population evolution may include a set of differential equations representing a time evolution of the plurality of cell population concentrations for the respective plurality of cell populations. For a particular therapeutic agent, the set of differential equations of the model of cell population evolution may depend on dose-response characteristics for that therapeutic agent. In some cases, the model of cell population evolution may model concentration changes among cell populations in the plurality of cell populations. It may, alternatively or additionally, model birth of a new cell population, and/or death of a cell population in the plurality of cell populations.

In some embodiments, the therapeutic agent may be a small molecule, a protein, a nucleic acid, gene therapy, a drug approved by regulatory approval agency, and/or a biological product approved by a regulatory approval agency. In some embodiments, the techniques described herein may further include administering one or more therapeutic agents to the subject in accordance with the dosage plan.

Some embodiments provide for a computer-implemented method for training a statistical model to determine a dosage plan for administering one or more therapeutic agents to a subject. The method may involve: (1) generating training data for training the statistical model using information specifying initial cell population concentrations for respective cell populations, and a model of cell population evolution; (2) training the statistical model using the training data to obtain a trained statistical model; and (3) storing the trained statistical model.

In some embodiments, the method may further comprise: (4) accessing information specifying, for the subject, cell population concentrations for the respective cell populations; and (5) determining, using the trained statistical model and the cell population concentrations, the dosage plan for administering one or more therapeutic agents to the subject, wherein the dosage plan may include one or more concentrations of the one or more therapeutic agents to be administered to the subject at different times. In some embodiments, each cell population may have respective dose-response characteristics different from dose-response characteristics of other cell populations. For example, different cell populations may be associated with respective dose-response curves that are different from one another (e.g., according to any suitable measure of difference between two dose-response curves).

In some embodiments, the statistical model may be trained using a reinforcement learning technique. In some embodiments, the statistical model may be trained using an actor-critic reinforcement learning technique. In some embodiments, the statistical model may be trained using a deep deterministic policy gradient (DDPG) algorithm.

In some embodiments, the training data may be generated using stochastic simulations of the model of cell population evolution. The model of cell population evolution may include a set of differential equations representing a time evolution of the initial cell population concentrations for the respective cell populations. In some embodiments, the one or more therapeutic agents include a first therapeutic agent, wherein the set of differential equations depends on dose-response characteristics for the first therapeutic agent.

As described herein, the techniques developed by the inventor(s) may be applied to in vitro biological samples. Accordingly, some embodiments provide for a method for performing controlled evolution of cells within a biological sample. The method may involve: (A) accessing information specifying cell population concentrations for respective cell populations in the biological sample; (B) using a trained statistical model and the cell population concentrations to determine concentrations of one or more agents to be administered to the biological sample; and (C) administering the one or more agents to the biological sample in the determined concentrations. In some embodiments, the acts (A), (B), and (C) may be performed repeatedly, for example, by automated lab machinery.

In some embodiments, the statistical model may be trained using training data generated using a model of cell population evolution. In some embodiments, the training data may be generated using stochastic simulations of the model of cell population evolution. In some embodiments, the model of cell population evolution includes a set of differential equations representing a time evolution of the plurality of cell population concentrations for the respective plurality of cell populations. In some embodiments, the one or more agents include a first agent, and the set of differential equations depends on dose-response characteristics for the first agent. In some embodiments, the trained statistical model includes a deep neural network model.

Following below are more detailed descriptions of various concepts related to, and embodiments of machine learning techniques for determining therapeutic agent dosages. Various aspects described herein may be implemented in any of numerous ways. Examples of specific implementations are provided herein for illustrative purposes only. In addition, the various aspects described in the embodiments below may be used alone or in any combination, and are not limited to the combinations explicitly described herein.

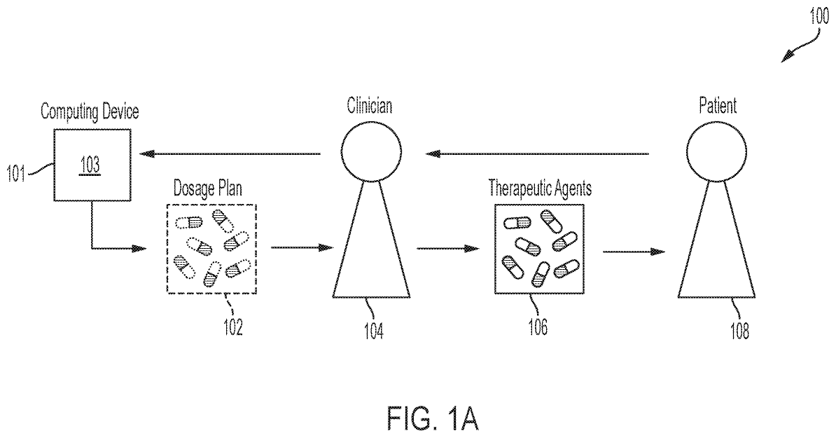

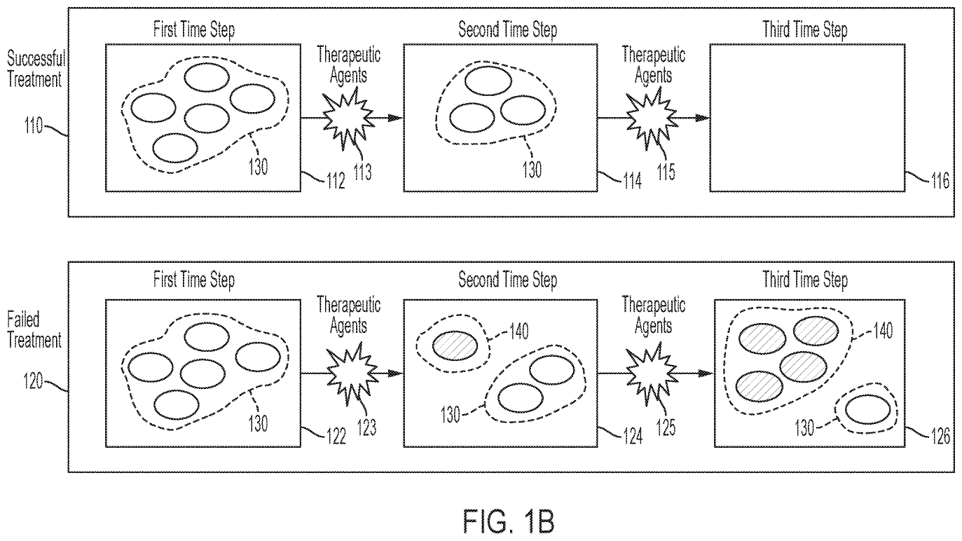

FIG. 1A is a system diagram showing an exemplary clinical setting 100 in which some of the techniques described herein may be employed. As shown in FIG. 1A, a clinician (e.g., clinician 104) may use the technique(s) developed by the inventor(s) to determine an appropriate treatment for a patient (e.g., patient 108). In setting 100, for example, clinician 104 may provide information about a patient 108 as input to computing device 101, which in turn may execute trained statistical model 103, to produce output indicating a dosage plan 102. The computing device 101 may provide this output to clinician 104 (e.g., via a display, by generating a report and sending that to the clinician through e-mail or any in other suitable way). This may occur immediately, or after a time delay of, for example, several minutes, several hours, or any other suitable amount of time. The clinician 104 may review the dosage plan 102 and determine one or more subsequent steps. For example, the clinician 104 may determine that patient 108 is to be treated in accordance with the dosage plan, and may administer one or more therapeutic agents 106 to the patient 108 and/or instruct patient 108 for what therapeutic agent to take and when. As another example, the clinician 104 may determine to not follow the course of treatment in the dosage plan. As yet another example, the clinician 104 may provide different input to the trained statistical model to generate a different dosage plan.

In the illustrated setting 100, the trained statistical model 103 is executed on computing device 101, which may be co-located with the clinician 104 (e.g., it may be in the clinician's office or practice or office building). In other embodiments, trained statistical model 103 may be executed by one or remote computing devices with which computing device is configured to communicate (e.g., via any suitable network connection). In some such embodiments, computing device 101 may provide, to a remote device (or devices) configured to execute the trained statistical model, information to use for forming input to the trained statistical model, and may receive, from the remote device(s), output from the trained statistical model.

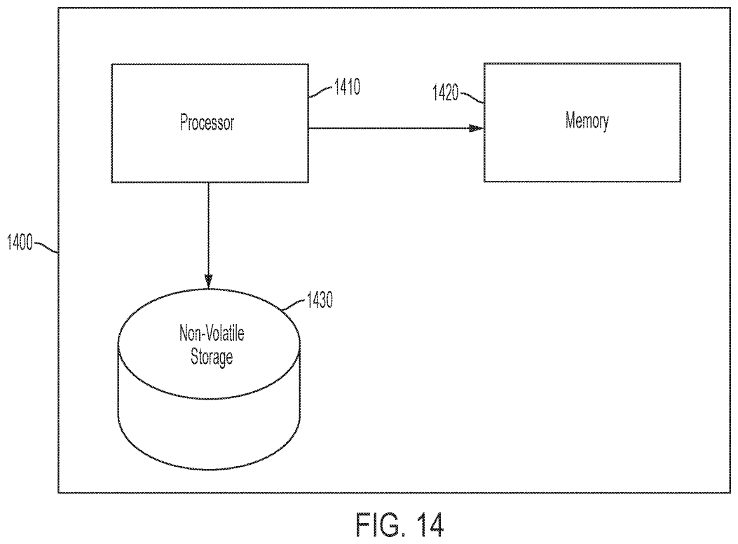

In some embodiments, computing device 101 may include one or multiple computing devices each of which may be embodied in a computer system such as the one described herein with respect to FIG. 14 below. Generally, computing device 101 may be any device configured to execute the trained statistical model and/or communicate with such a computing device or devices.

In some embodiments, the dosage plan 102 may comprise information specifying concentrations of one or more therapeutic agents to be administered to a subject as part of treatment, as well information specifying time intervals at which these concentrations of therapeutic agents are to be administered. In some embodiments, dosage plan 102 may include only information specifying a next dosage for the treatment: for example, the dosage plan 102 may indicate concentrations of therapeutic agents to be applied at a next time step of the treatment, which may, for example, occur immediately, or may occur at a later time, such as several days, weeks, or months from the time at which the dosage plan was determined.

In some embodiments, the dosage plan may indicate information specifying a sequence of dosages to be administered at multiple respective times. Moreover, the concentrations of the therapeutic agent(s) 106 may be discrete (e.g., selected from a small number or large number of therapeutic agents, which may be administered in forms such as pills, injections, etc.) or they may be continuous (e.g., selected from a continuous range of values, such as for intravenous administration). It should also be appreciated that the administration of therapeutic agents 106 need not involve direct interaction between the clinician 104 and the patient 108, but may occur automatically (e.g., via an automated intravenous infusion mechanism) or otherwise without the clinician 104 being personally involved.

In some embodiments, the therapeutic agent(s) 106 may take many forms besides the illustrated example of pills, and may comprise, for example, a small molecule, a protein, a nucleic acid, gene therapy, a drug approved by regulatory approval agency, or a biological product approved by a regulatory approval agency.

As further shown in FIG. 1A, the computing device 101 may also receive from the clinician 104 information which the clinician obtained from the patient 108. For example, the clinician 104 may obtain a biological sample from the patient 108, determine cell population concentrations for cell populations within that biological sample, then provide these cell population concentrations to computing device 101. These cell population concentrations may be accessed by the computing device 101 and provided as input to the trained statistical model, which may be a neural network model trained with reinforcement learning techniques such as those described herein, to produce the dosage plan. As another example, the clinician may use a biological sample obtained from the patient 108 to determine dose-response characteristics of cell populations present in the biological sample, such as resistance/susceptibility to a particular therapeutic agent. Regardless of whether or what information the clinician 104 obtains from patient 108, the information may be collected from the patient in a variety of ways, which may or may not require direct interaction between the clinician 104 and patient 108.

Not all of the elements depicted in the clinical setting of FIG. 1A need to be present and/or interact in the manner shown. For example, in some embodiments, therapeutic agents 106 may not be applied to patient 108. This might occur, for example, if the clinician 104 determines that a particular dosage plan 102 involves therapeutic agents having an unacceptably high toxicity in the required concentrations, or deems the dosage plan 102 unsatisfactory for another reason. In some embodiments, the process shown in FIG. 1A may be entirely automated, such that there may be no clinician 104. For example, in an intensive care unit (ICU) setting, the entire process may be carried out by automated equipment including, e.g., intravenous administration of therapeutic agents and collection of biological samples from the patient 108.

In some embodiments, aspects of the technology shown in FIG. 1A may be applied in an in vitro setting rather than the illustrated clinical setting 100. This may occur, for example, in a laboratory as part of a controlled evolution experiment, such as a bacterial evolution experiment or a mammalian cell line experiment. In such cases, an experimenter, rather than clinician 104, may interact directly with a biological sample instead of patient 108. As in the clinical setting, the experimenter may interact with computing device 100 to provide information obtained from the biological sample (e.g., cell population concentrations) to the computing device, and may receive from the computing device a dosage plan 102. In this alternative embodiment, the dosage plan 102 may include information specifying concentrations of agents to be administered to the biological sample, and information specifying time intervals at which these concentrations of agents are to be administered. As described above with respect to the clinical setting, the agents may take many forms, and may be administered in a variety of ways. Particularly in the in vitro case, the agents may be administered, for example, with an automated liquid handling system. More generally, for techniques described herein with respect to the clinical setting, corresponding techniques also apply to the in vitro setting, mutatis mutandis.

FIG. 1B is a diagram showing illustrative treatments being applied to cell populations. These treatments may, for example, be treatments such as those developed within the clinical setting of FIG. 1A, but in general may be treatments developed in any suitable manner. The figure shows two exemplary treatments: a successful treatment 110, and a failed treatment 120. The illustrated treatments are applied to respective cell environments containing two different cell populations, 130 and 140, having varying cell population concentrations. In both treatments, the goal may be to reduce the cell population concentrations for the depicted cell below an acceptable threshold. In some cases, this may entail completely eliminating the depicted cell populations from the environments.

The successful treatment 110 takes place across a first time step 112, a second time step 114, and a third time step 116. The time steps 112, 114, and 116 may take place across seconds, minutes, hours, or any other suitable time interval depending on the nature of the cell environment and any other constraints of the system. At the first time step 112, the cell population 130 is shown having a high cell population concentration within the cell environment. Although this is shown in the figure as five individual cells within the cell population 130 at time step 112, cell population concentrations in real-world cell environments may be on the order of 10⁶ or 10⁷ cells/mL, for example, and may be expressed in any suitable units. After time step 112, therapeutic agents 113 are applied, leading to the second time step 114. At time step 114, the cell population concentration of cell population 130 has been reduced, as indicated by the reduced number of cells shown within the cell population. This may be a result of the application of therapeutic agents 113, which may interact with the cell population 130 to reduce the birth rate or increase the death rate of cells within the cell population, causing the corresponding cell population concentration to decrease. After time step 114, therapeutic agents 115 are applied, leading to the third time step 116. The therapeutic agents 115 need not be the same as therapeutic agents 113. In some embodiments, it may in fact be desirable that different therapeutic agents be used, so as to target particular cell populations. At time step 116, cell population 130 has been successfully eliminated (or, in some cases, reduced below an acceptable threshold), and treatment 110 is complete.

The failed treatment 120 takes place across a first time step 122, a second time step 124, and a third time step 126. As above, the time steps 122, 124, and 126 may take place across seconds, minutes, hours, or any other suitable time interval depending on the nature of the cell environment and any other constraints of the system. At the first time step 122, the cell population 130 is shown having a high cell population concentration within the cell environment. After time step 122, therapeutic agents 123 are applied, leading to the second time step 124. At time step 124, the cell population concentration of cell population 130 has been reduced, as indicated by the reduced number of cells shown within the cell population.

However, cell population 140 has a non-zero cell population concentration at step 124, as indicated by the cell depicted within cell population 140 at time step 124. The emergence of cell population 140 may, for example, be a result of random mutations within cell population 130. For example, if the therapeutic agents 123 reduce the birth rate only for some cells among cell population 130, cells having lowered susceptibility to the therapeutic agent will be more likely to reproduce, eventually resulting in the emergence of a new cell population 140 that may exhibit resistance to the therapeutic agents 123. In general, as described herein, the emergence of new cell populations within a cell environment is a stochastic process driven by complex biological factors and mechanisms. Regardless of how cell population 140 may have emerged, after time step 124, therapeutic agents 125 are applied, leading to the third time step 126. At time step 126, neither cell population 130 nor cell population 140 have been eliminated, as desired, and the cell population concentration for cell population 140 has in fact increased. This may occur, for example, because cell population 140 may have limited susceptibility to the therapeutic agents 123 and/or 125. Regardless, since the depicted cell populations 130 and 140 have not been eliminated by time step 126, treatment 120 has failed.

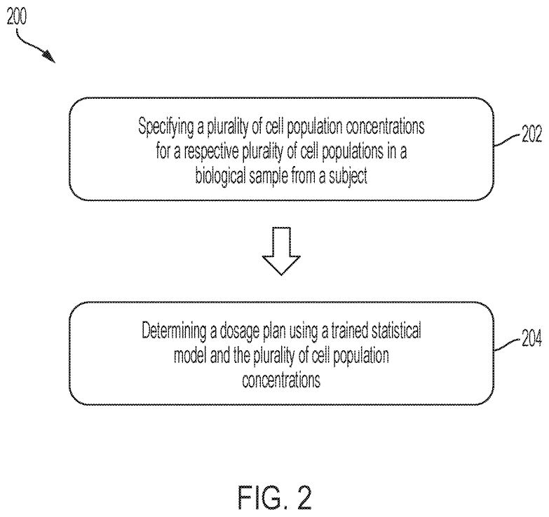

FIG. 2 is a flowchart of an illustrative method 200 for determining a dosage plan for administering at least one therapeutic agent to a subject that may be applied in clinical or in vitro settings, in accordance with some embodiments of the technology described herein. As should be appreciated from the foregoing description of FIG. 1A, techniques from one setting may be applied to the other, mutatis mutandis.

At block 202, a plurality of cell population concentrations for a respective plurality of cell populations in a biological sample from a subject are specified. Cell population concentrations may be specified in any suitable manner, such as in cells per mL or other units of concentration. According to some embodiments, cell population concentrations may be determined based on observations collected from the biological sample. In the clinical setting, this may entail taking and analyzing the biological sample from a subject, who may, for example, be a human patient undergoing treatment. In the in vitro setting, the biological sample may be analyzed directly to obtain observations, or a sample may be obtained and analyzed. In either setting, the observations collected may include observations about the cell populations present in the cell environment of the biological sample.

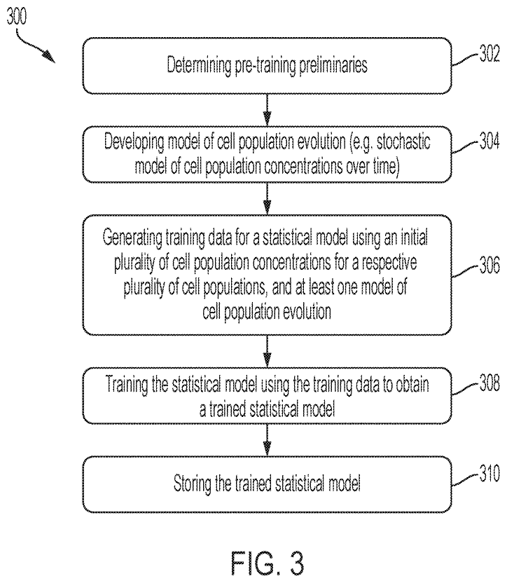

At block 204, a dosage plan is determined with a trained statistical model and the plurality of cell population concentrations specified at block 202. According to some embodiments, the cell population concentrations may be provided as input to the trained statistical model, which may produce, as its output, the dosage plan. The operation of the statistical model, including the manner in which the trained statistical model may be developed, is described herein at least with respect to FIGS. 3, 4, and 7 below. As described herein including with respect to FIG. 1A, the dosage plan includes information specifying concentrations of agents to be administered, and time intervals at which those concentrations of agents are to be administered. In some embodiments, the agents specified in the dosage plan may be administered based on that dosage plan. As described herein including with respect to FIG. 1A, the administration of agents may be performed in a number of ways, which may involve, for example, administration by a clinician, an experimenter, or a mechanism for automatically administering the agents. In some embodiments, after the agents are administered, the method 200 may return to block 202. That is, a treatment applied according to the depicted method may be a repeated method, involving repetitive steps of collecting observations, specifying cell population concentrations, determining a dosage plan, and applying agents according to that dosage plan. This method may repeat, for example, until the experiment or treatment is successful, or until the experiment or treatment fails.

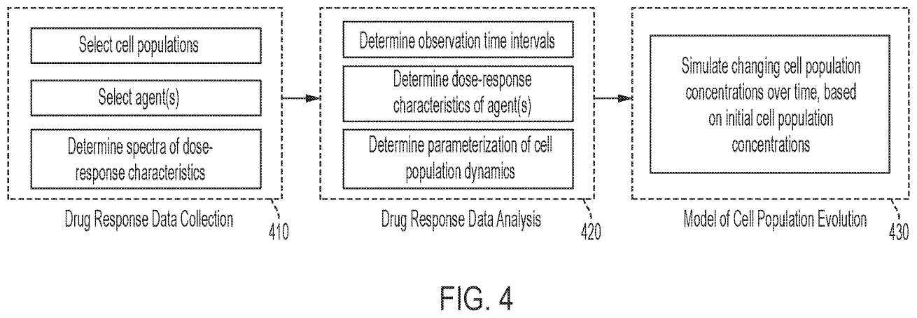

FIG. 3 is a flowchart of an illustrative method for training a statistical model to determine a dosage plan for administering at least one therapeutic agent to a subject. At block 302, pre-training preliminaries may be determined. These pre-training preliminaries may include information specifying the cell populations of interest. For example, the cell populations may be specified according to their differing dose-response characteristics. As described herein at least with respect to FIG. 4, determining the pre-training preliminaries may include determining a spectrum of dose-response characteristics for the cell populations of interest.

The pre-training preliminaries may further include information specifying the therapeutic agents that may be used in the dosage plans determined by the statistical model. Additional information about the therapeutic agents may also be included in the pre-training preliminaries. For example, the pre-training preliminaries may indicate preferences for certain therapeutic agents over other therapeutic agents. These preferences may be provided by a clinician, and may be determined, for example, by the toxicity or clinical effectiveness of certain therapeutic agents relative to other therapeutic agents. In some cases, hard limits may also be provided by a clinician such as, for example, maximum dosing limits.

The pre-training preliminaries may also include information specifying the time intervals that may be used in the dosage plans determined by the statistical model. These time intervals may be determined by a clinician, for example, and may reflect the time intervals at which observations become available and/or the time intervals at which the concentrations of therapeutic agents being administered can be altered.

In a clinical setting, the pre-training preliminaries may also include additional information specified by the clinician. For example, the clinician may determine and provide, as part of the pre-training preliminaries, additional quantitative and qualitative information on drug toxicity, cost, and preference. The clinician may similarly obtain and provide information including: units at which drug is administered (e.g., if orally administered); the maximal allowable treatment duration; the importance of shortened treatment duration; the relevant pharmacokinetic parameters for translating in vitro concentrations to relevant in vivo concentrations; and feasible clinical sample time intervals.

At block 304, a model of cell population evolution may be developed based on the pre-training preliminaries determined at block 302. This model of cell population evolution may be a stochastic and mechanistic model that may use, for example, stochastic differential equations to predict the evolutionary trajectories of cell populations. In particular, the model of cell population evolution may be designed to predict changing cell population concentrations over time, based on initial cell population concentrations. The model of cell population evolution may be parametrized by some or all of the pre-training preliminaries determined at block 302. A process for developing a model of cell population evolution is described herein at least with respect to FIG. 4.

At block 306, training data for the statistical model is determined using an initial plurality of cell population concentrations, which may vary or be set at different levels, for example, and the model of cell population evolution developed at block 304. The training data may include simulated cell population concentrations generated by the model of cell population evolution based on input cell population concentrations. The contents and operation of the model of cell population evolution are described herein at least with respects to FIGS. 4-6.

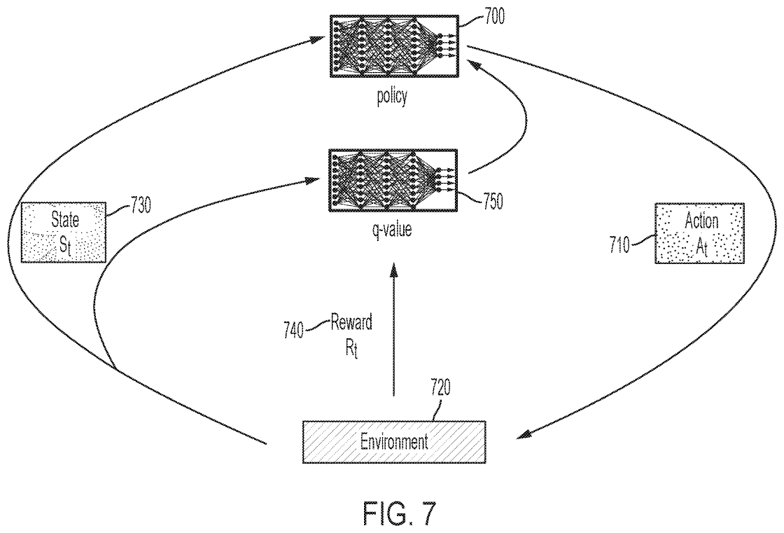

At block 308, the statistical model is trained to determine a dosage plan for administering therapeutic agents, based on the training data generated at block 306. Although not shown in the figure, training the statistical model at block 308 may involve returning to block 306 to generate additional training data. In particular, as part of training the statistical model using reinforcement learning techniques, current cell population concentrations associated with the statistical model at block 308 may be provided as input to the model of cell population evolution at block 306, allowing the model of cell population evolution to produce simulated cell population concentrations for a next time step of the statistical model at block 308. It is worth noting here that while the time steps of the statistical model correspond to the time intervals for observations/doses determined as part of the pre-training preliminaries at block 302, the model of cell population evolution may simulate cell population changes at different, significantly smaller time intervals. For example, the time steps of the statistical model (and corresponding dosage plans) may be on the order of multiple hours or days, while the model of cell population evolution may simulate changes in cell populations concentrations on the order of seconds or milliseconds. Regardless, the time steps need not be fixed for either the statistical model or the model of cell population evolution. The training process for the statistical model is described herein at least with respect to FIGS. 7-10 below.

At block 310, the trained statistical model is stored. According to some embodiments, the trained statistical model may be stored in non-transitory computer-readable storage media, such as is described with respect to FIG. 14 below. Storing the trained statistical model may entail storing weights of the statistical model that may be determined as part of training.

Note that in some embodiments, determining the pre-training preliminaries at block 302 and developing the model of cell population evolution at block 304 need not occur as part of method 300. For example, it may be the case that the pre-training preliminaries and the model of cell population evolution have already been established prior to the actual training of the model in steps 306 through 310. (e.g., by a clinician or experimenter other than the one training the statistical model)

FIG. 4 is a diagram illustrating developing a model of cell population evolution. Since the development of a model of cell population evolution may be part of the training process for the statistical model, as previously described, the process depicted in FIG. 4 may overlap with or correspond to blocks 302, 304, and 306 of FIG. 3.

In some embodiments, a model of cell population evolution may be any suitable model that specifies how concentrations of cell populations vary in time. In some embodiments, the model of cell population evolution may be a model of biological natural selection; however, the model is not limited in this respect, and may be any appropriate, scenario-relevant model that describes the temporal trajectory of cell populations. In some embodiments, the model of cell population may be specified using one or multiple differential equations (e.g., stochastic differential equations) and/or difference equations. In some embodiments, the model of cell population may be specified in terms of one or more parameters non-limiting examples of which include birth rate, death rate, carrying capacity of the cell environment, cell population concentrations, number of cell populations, extent of resistance of cell populations to a therapeutic agent, simulation step time, probability of mutations, etc. In some embodiments, the model of cell population evolution of a cell population may depend on the dose-response characteristics (e.g., as may be captured by a dose-response curve) of the cell populations.

Returning to FIG. 4, at block 410, drug response data collection is performed. This data collection may include selecting cell populations, selecting agents, and determining a spectrum of dose-response characteristics for the selected cell populations and agents. Selecting cell populations may include determining cell populations of interest for a particular clinical or experimental goal. For example, an experimenter may be interested in controlling the evolution of E. coli, or a clinician may wish to develop a treatment for S. aureus. Selecting agents may similarly include determining the agents most relevant to a particular clinical or experimental goal. In the example of controlling the evolution of E. coli, the experimenter may determine that trimethoprim, ciprofloxacin, and imipenem are the agents to be used in the experiment. In the example of treating S. aureus, the clinician may determine that methicillin, oxacillin, flucloxacillin, vancomycin, and clindamycin are the therapeutic agents usable in treatment. In practice, many more agents may be selected and incorporated into the model of cell population evolution and the corresponding statistical model.

Regardless of the cell populations and agents selected, at block 410, a spectrum of dose-response characteristics for the selected cell populations and agents may be determined. In practice, spectra of dose-response characteristics may be determined experimentally, by allowing cell populations to evolve in vitro under different agent concentrations. Continuing the example of the experimenter interested in controlling the evolution of E. Coli, this may involve the experimenter taking a set of E. coli samples and running in vitro evolution experiments to obtain the spectrum of potential responses to each of trimethoprim, ciprofloxacin, and imipenem. Learning the spectrum of dose-response characteristics may allow the model of cell population evolution to define the cell populations based in part on where their dose-response characteristics fall within the determined spectrum of dose-responses characteristics. Defining cell populations in this way enables the model of cell population evolution, and, consequently, the statistical model, to “bin” any new variants that arise among the cells according to their dose-response characteristics, without needing to specifically characterize all possible and potential mutations in advance. Preferably, with enough training data and an appropriate initial spectrum of dose-response characteristics, it will be the case that new cell populations that arise during training or application of the model will not exhibit dose-response characteristics significantly different from those already observed.

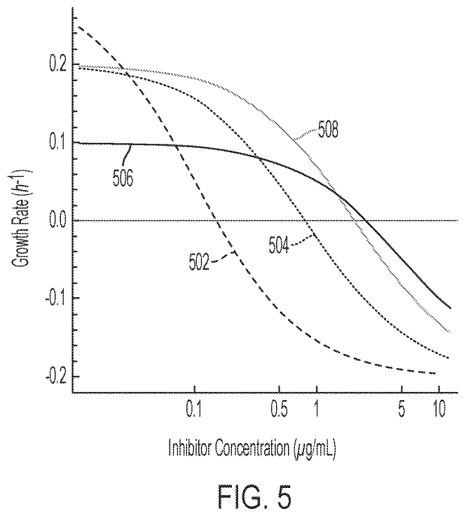

At block 420, additional drug response data analysis may be performed. This data analysis may include determining observation time intervals, determining specific dose-response characteristics for the cell populations/agents, and determining a parametrization of cell population dynamics. As described herein, the time intervals for observation may be determined by a clinician, and may be constrained by a variety of factors (e.g., real-world constraints of the system, such as patient or clinician needs, etc.). Determining specific dose-response characteristics for the cell populations and agents may involve establishing, within the spectrum determined at block 410, the particular ranges of dose-response characteristics that define the “bins”. Determining the dose-response characteristics at block 420 may also include determining specific dose-response curves (see, for example, FIG. 5 and the corresponding description) for the particular cell populations and agents.

Also at block 420, a parameterization of cell population dynamics may be determined. In general, there are many ways that cell population dynamics may be parameterized. In some embodiments of the techniques described herein, the evolution of the cell populations may be described with a system of stochastic differential equations (SDEs) that may be parameterized by parameters including, for a cell population i and therapeutic agents α: cell birth rate βᵢ, cell death rate δᵢ, and therapeutic agent resistance parameter ρ_{i,α}, which represents the extent of resistance cell population i exhibits with respect to therapeutic agent α. As part of developing the model of cell population evolution, a functional dependence of the growth rate on these parameters and the therapeutic agent(s) concentrations for a particular cell population may be determined.

In some embodiments, defining the “bins” for cell populations described above may involve specifying ranges for each of these parameters, not just the therapeutic agent resistance parameters. As an example, consider two cell populations with identical concentrations at time t=0 and suppose that their evolution is simulated for a period of time τ. If, averaged across some pre-specified number of runs of the evolutionary simulation (e.g., 20), the difference between their final cell population concentrations at the conclusion of an observation time τ exceeds some acceptable threshold, then the two cell populations should be in separate bins. Thus cell populations falling within the same bin, based on these parameters, should result in a sufficiently-similar trajectory over a chosen time interval, where longer time intervals may involve higher thresholds.

At block 430, the model of cell population evolution simulates changing cell populations over time based on initial cell population concentrations. This may involve providing the cell population concentrations as inputs to the set of SDEs describing the model of cell population evolution. Additional inputs to the model of cell population evolution may be required, such as the concentrations of therapeutic agents being applied in the cell environment. As described herein, the time steps simulated by the model of cell population evolution may be on the order of seconds, milliseconds, or any other, not necessarily fixed, length of time. The output of the model of cell population evolution at a given time step may be updated cell population concentrations for the next time step of the simulation. As described herein, for a single observation/model time step, many simulation time steps may be performed.

According to some embodiments, mutations may be modeled within the model of cell population evolution as random perturbations influencing the SDEs describing the model. Mutations may be set to occur at a particular rate within the model of cell population evolution, in order to accurately model the rate at which mutations may be expected to arise in real-world cell environments. In some embodiments, the rate may vary, and in general it need not be a fixed rate. In some embodiments, modelling mutations may include modelling the birth of a cell population. Within the model of cell population evolution, the birth of a cell population may involve a cell population with a cell population concentration of zero having, at a subsequent time step, a cell population concentration above zero.

More details regarding exemplary SDEs and their parametrization may be found in the “Stochastic Modeling and Simulation of Cell Population Evolution” below.

FIG. 5 is a chart showing exemplary dose-response characteristics for some cell populations. In particular, the chart indicates dose-response curves for four cell populations 502, 504, 506, and 508. These dose response curves are plotted over an x-axis indicating the concentration of the therapeutic agent (shown as “Inhibitor concentration” in the chart, typically given as cell numbers (or CFU, colony-forming units) per unit volume, which may be a count of viable cells), and a y-axis indicating growth rates for the plotted cell populations. In the illustrated chart, cell population 502 has the highest growth rate when the concentration of the therapeutic agent is zero. This may imply that cell population 502 should be taken as the baseline cell population, which may be dominating the cell environment at the onset of simulation, since its growth rate is the highest in the absence of the therapeutic agent. In the illustrated chart, cell populations 506 has the lowest growth rate when the concentration of the therapeutic agent is zero. However, the dose-response curve for cell population 506 indicates that it may exhibit the most resistant to the therapeutic agent being administered: in particular, the growth rate for cell population 506 is the last to cross from a positive growth rate to a negative growth rate under increasing concentrations of the therapeutic agent.

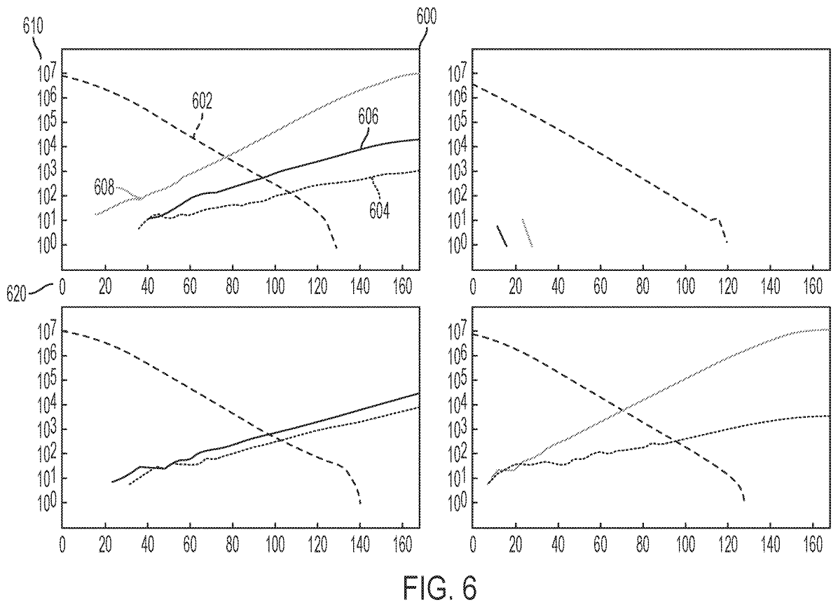

FIG. 6 is a chart showing sample cell population evolution simulations, such as may be produced by a model of cell population evolution. The horizontal and vertical axes represent, respectively, time in hours and cell population concentrations. In the labelled chart, the x-axis 610 represents time in hours, and the y-axis 620 represents cell population concentrations. Evolutionary trajectories for each of the four cell populations 502, 504, 506, and 508 shown in FIG. 5 are shown as 602, 604, 606, and 608 in FIG. 6. In the illustrated charts, the initial state in any simulation was a population comprised entirely of the most susceptible baseline cell population with a population concentration between 10⁶-10⁷ cells/mL (random initialization in discrete steps of 10⁶), but mutations were allowed from the first time step and could occur at any subsequent time step (4 hour intervals). Even when a cell population is sufficiently strong to survive the administered dosage, random demographic fluctuations may eliminate its nascent population, as shown in several of the plots. Even when no mutations occur, variability in the initial size of the population as well as demographic randomness can lead to significantly different extinction times for the susceptible cell population.

FIG. 7 is a is a diagram illustrating aspects of training a statistical model using reinforcement learning. In the depicted example, the goal of training is to learn a policy 700 that determines actions 710 to be taken within an environment 720, based on the state 730 of the environment. According to the aspects of training shown in the figure, the policy 700 may be optimized under set optimality guidelines, which may be specified by the reward 740.

In general, the goal of reinforcement learning approaches may be to learn an optimal decision-making policy through an iterative process in which the policy is gradually improved by sequential tuning of parameters either of the policy itself or of a value (e.g., an action-value function, shown in the figure as Q-function 750) indicative of the policy's optimality. The data on which learning is done may be supplied in the form of transitions from a previous state s of the environment to the next state s′ that occurs probabilistically (in stochastic environments) when a certain action a is chosen. Each such transition may be assigned a reward r, and reward maximization may drive the optimization of the policy.

Learning can be done on a step-by-step basis or episodically, where a single episode consists of multiple steps of transitions. In the case of determining dosage plans, as considered here, an episode may be a single-patient full course of treatment or, for the in vitro case, a single control experiment. An episode may conclude when either the maximal treatment time has been reached or all the cell populations targeted by the treatment have been eliminated, whichever comes first. Episodes are thus finite but can be long, with drugs potentially being administered at discrete time steps over a continuous range of dosages. At each time interval, a decision is made on which therapeutic agents at what concentrations should be administered based on observations of the current state of the cell environment.

More specifically, in the case of training a statistical model for determining a dosage plan, the state 730 of environment 720 may specify the cell population concentrations of cell populations within the environment 720. Additional information may also be specified as part of the state, including, for example, growth rates for the cell populations and/or overall growth rates for the targeted cell populations, such as may be determined by a model of cell population evolution. The state need not include this information explicitly and may represent this information in any suitable manner (e.g., as a vector or another data structure). The corresponding actions 710 may take the form of concentrations of therapeutic agents to be administered within the environment 720. That is, when the policy 700 determines a particular action 710, it may be specifying concentrations of a therapeutic agent to be applied at a next time interval based on the state 730. The system shown in FIG. 7 is thus continuous in its state space (cell population concentrations), involves time-dependence and potentially high stochasticity, can be high-dimensional due to the large number of possibly-occurring mutant cell populations, and involves one or multiple therapeutic agents that can take a continuous range of values (e.g., if administered intravenously). Thus for reasons of mechanical operability and medical explainability, a deterministic policy 700 may be desirable in this case.

With regards to the reward 740, an optimal policy 700 may be one that provides a successful dosage plan with the lowest expected cumulative dosing (e.g., lowest total concentration of therapeutic agent(s) applied) over the course of the treatment. As described herein including with respect to FIG. 1B, recall that a treatment may be considered successful if all cell populations targeted by the treatment are eliminated (or reduced below an acceptable threshold) by the final time step of treatment. In addition to treatment success and cumulative therapeutic agent concentrations, other factors may also be incorporated into the reward 740. For example, the reward 740 may include penalty weights that may be applied to reflect preferences for certain therapeutic agents over others (e.g., a last-line drug will incur a higher penalty that that of a first-line drug). If an episode fails, the reward 740 may include a penalty that is proportional to the extent of failure (e.g., the penalty increases with a higher ratio of remaining cell population concentrations to the initial cell population). In some embodiments, the reward 740 may also provide a guiding signal in the form of potential-based reward shaping. Further details about reward assignment are provided in the “Exemplary Statistical Model” section below.

In the illustrated diagram, neural network-based function approximation is used to optimize the policy 700. However, any appropriate technique for optimization, including other machine learning techniques not limited to reinforcement learning, may be employed for this purpose. More particularly, with regards to FIG. 7, an actor-critic deep learning architecture is shown. According to some embodiments described herein, the DDPG architecture additionally includes target actor and critic networks, which for simplicity are omitted in FIG. 7. In FIG. 7, the illustrated architecture includes a Q-function 750. The q-value 750 may represent an action-value function that represents the optimality of an action taken from a given state 730. As part of determining an optimal policy 700, machine learning techniques may be used to approximate and improve the Q-function 750. In turn, the Q-function may enable the policy 700 to make decisions about which action is to be taken in a particular state.

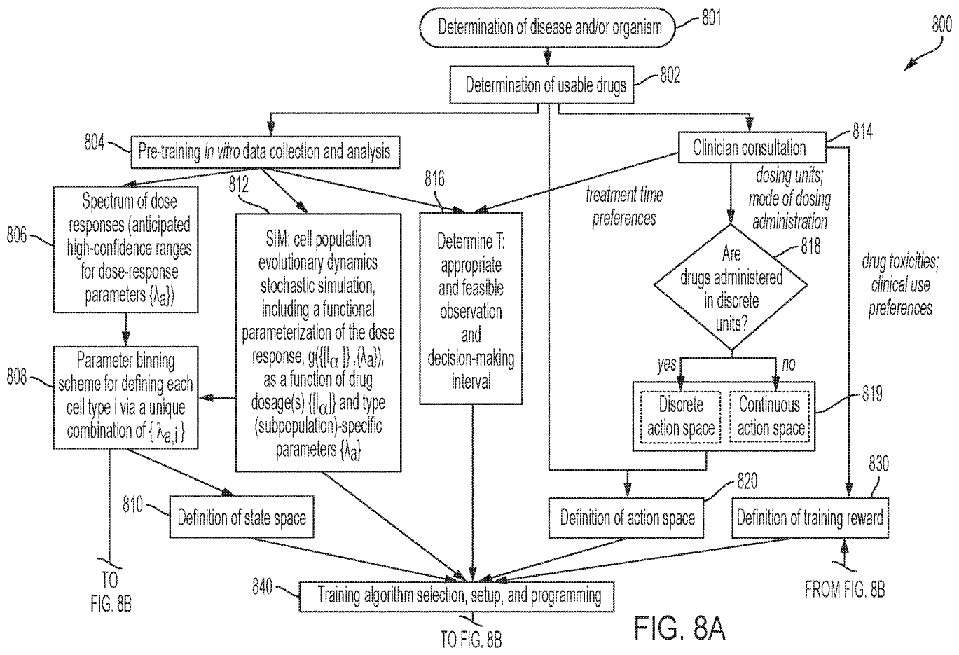

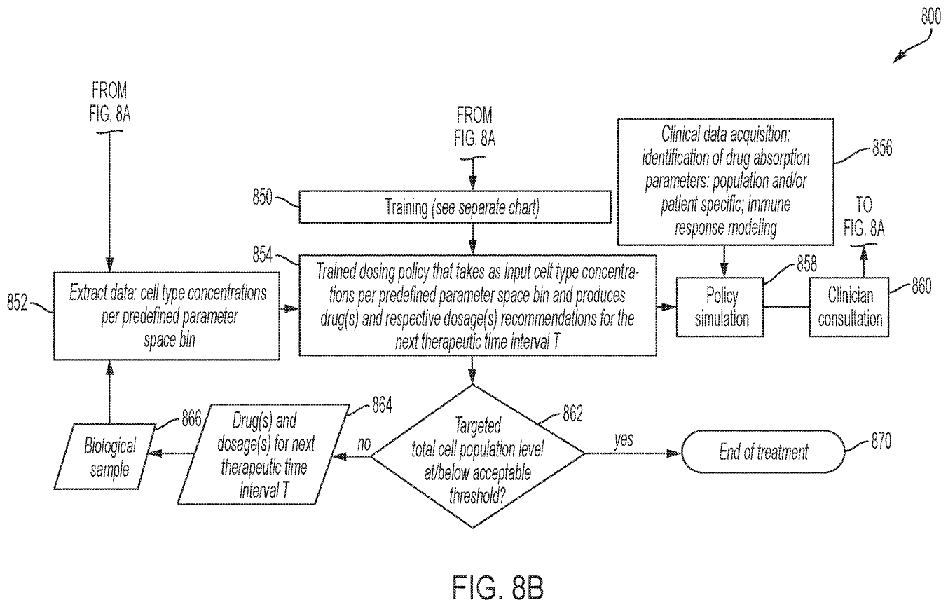

FIG. 8A-8B is a flowchart showing an illustrative process 800 for training a statistical model for determining a dosage plan, including the pre-training steps and the application of the trained statistical mode, in accordance with some embodiments of the technology described herein. Process 800 begins at block 801 with a determination of the disease and/or organism. In particular, this may be a determination of a disease and/or organism (e.g. cell populations) that is to be treated or controlled via therapeutic agents. For example, a clinician may wish to treat a disease caused by particular cell populations within a human patient. At block 802, a determination of usable drugs is made. In particular, the drugs may be therapeutic agents usable in the treatment of the disease determined at block 801. As with block 801, this determination may, in some embodiments, be made by a clinician. At block 804, process 800 performs pre-training in vitro data collection and analysis. The pre-training in vitro data collection and analysis may be carried out according to blocks 410 and 420 of FIG. 4, as described above. As shown in FIG. 8A-8B, pre-training may initially involve three aspects: determining a spectrum of dose-response characteristics at block 806, developing a model of cell population evolution at block 812, and determining time intervals at block 816. Each of these aspects may be as described above with respect to FIG. 4.

After a spectrum of dose-response characteristics has been determined at block 806, this information may be used in order to establish “bins” for cell populations, as described with respect to FIG. 4 above. As shown in FIG. 8A-8B at block 810, these bins may be used to define a state space, such as the state space described with respect to FIG. 7, for the statistical model. In particular, the states of the state space may be defined in terms of cell population concentrations, wherein each cell population is distinguished from other cell populations according to its bin (e.g., based on its parameters and corresponding evolutionary trajectory, as described above).

At block 812, a model of cell population evolution is developed. This may be, for example, according to the techniques described with respect to FIG. 4 above. At block 816, time intervals for the statistical model are determined. As shown in the FIG. 8A-8B, these time intervals may be based on clinician consultation 814 as well as pre-training in vitro data collection and analysis 804. That is, as described above, the time intervals for the statistical model may be determined by preferences or constraints established in advance by a clinician. These may include, for example, treatment time preferences.

In addition to helping determine the time intervals at block 816, the clinician consultation 814 may also help determine dosing information, such as dosing units and/or mode of dosing administration, which in turn may help define the action space for the statistical model. In particular, at block 818, it is determined based on the clinician consultation 814 whether the drugs being used in treatment are to be administered in discrete units. This informs the determination, at block 819, to define either a discrete or continuous action space for the statistical model. In some embodiments, the action space may be a mixed action space having both continuous and discrete actions (e.g. dosage concentrations). According to the determination at block 819, as well as the determination of usable drugs at block 802, the action space for the statistical model may be defined at block 820.

The clinician consultation 814 may also help determine drug toxicities and clinical use preferences, which may help define the training reward for the statistical model at block 830. An exemplary process for determining training reward is described herein at least with reference to FIG. 7 and the “Exemplary Statistical Model” section below.

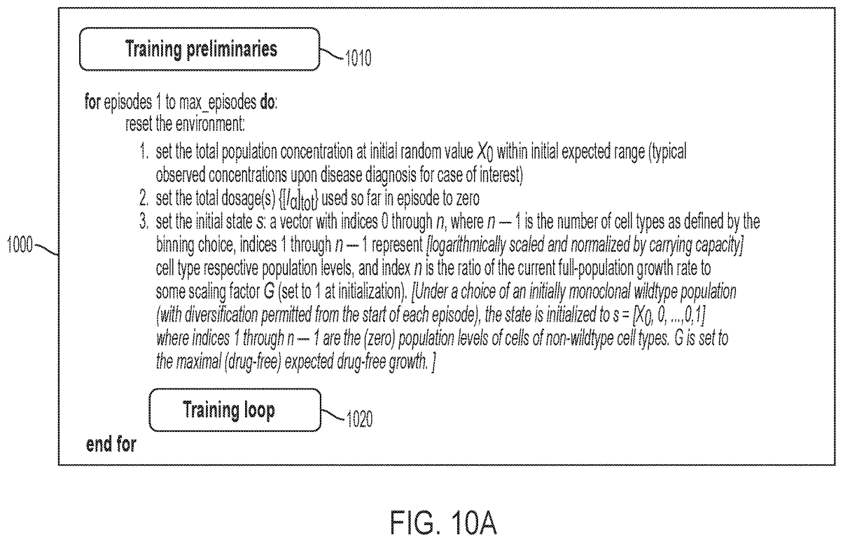

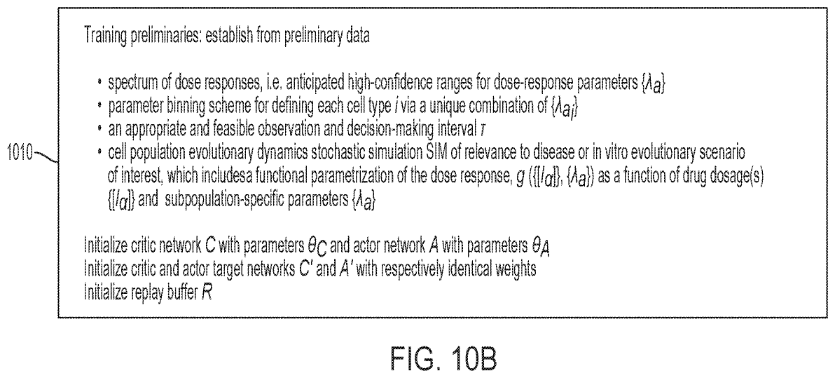

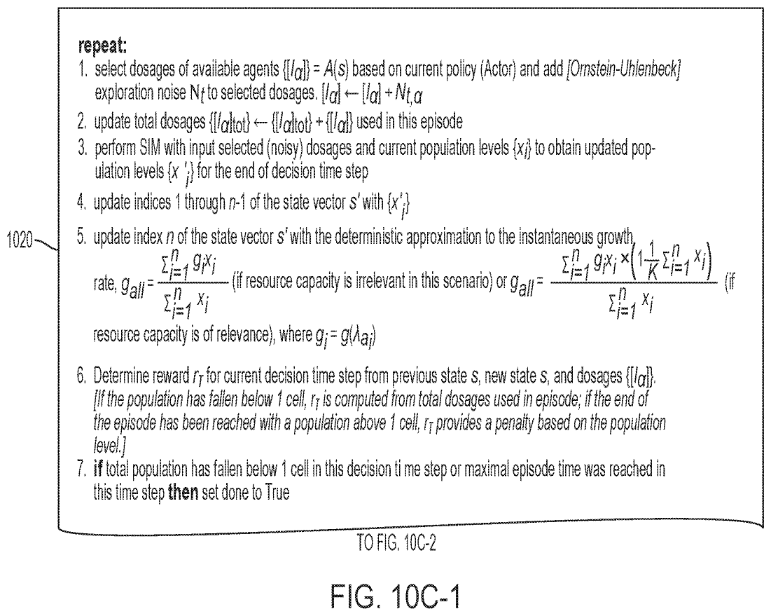

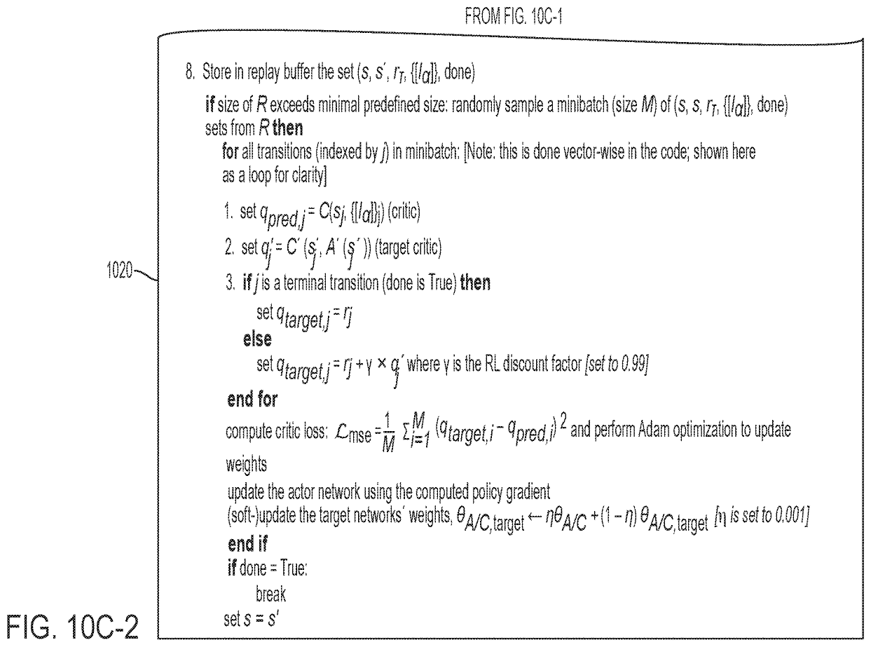

Based on the definition of the state space 810, the action space 820, and the training reward 830, as well as the model of cell population evolution 812 and the time intervals 816, at step 840, training algorithm selection, setup, and programming may be performed. This may include, for example, selecting a particular training algorithm to employ during training (e.g., a reinforcement learning algorithm, which may be an actor-critic reinforcement learning algorithm, which may employ Q-learning, deep deterministic policy gradient, or any other suitable machine-learning optimization techniques). Based on the selected training algorithm, a software implementation of the algorithm may be programmed and corresponding setup performed (see, e.g., FIGS. 10A-10C).

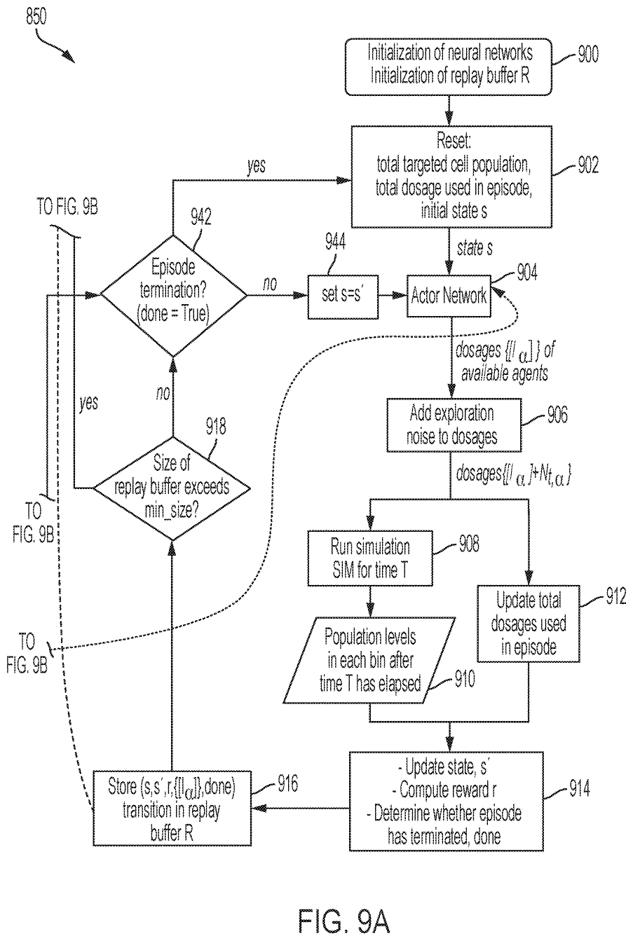

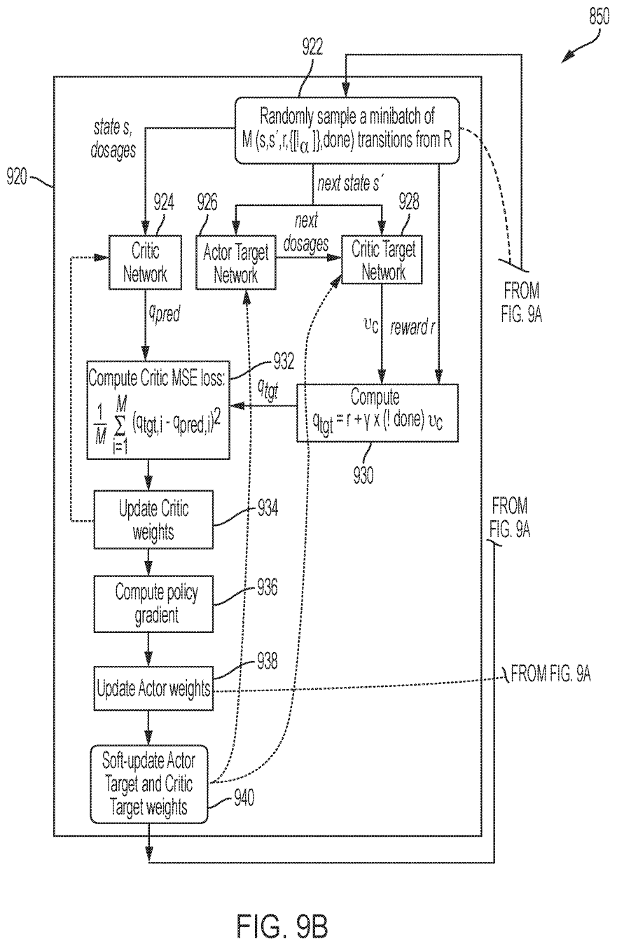

At act 850, the statistical model is trained. This is shown in the flowchart of FIG. 9A-9B, described herein.

At block 854, the trained statistical model is established. As shown in the figure, the trained statistical model takes as input cell population concentrations, such as may be extracted at block 852, and produces corresponding dosage recommendations (e.g., concentrations of therapeutic agents to be administered at the determined time interval). This may allow, in combination with clinical data acquisition at block 856, for policy simulation to be performed at block 858. The results of policy simulation may be used as part of another clinician consultation 860, the results of which may in turn be used to update the training reward at block 830 and, if necessary, may result in the retraining of the model.

At step 862, it is determined whether the cell population concentrations of the targeted cell populations are below an acceptable threshold. This may, for example, indicate whether the treatment thus far has been successful. In the case where the cell population concentrations are not below the acceptable threshold, then at block 864 therapeutic agents and corresponding concentrations (e.g., drugs and dosages) may be determined for the next time interval. These therapeutic agents may be applied to a biological sample at block 866, and updated data (e.g., cell population concentrations) may be extracted at step 852 to feed back into the trained model 854.

If, instead, the cell population concentrations are below the acceptable threshold, the treatment may conclude successfully at block 870.

FIG. 9A-9B is a flowchart showing an illustrative implementation of act 850 of process 800 for training a statistical model for determining a dosage plan, in accordance with some embodiments of the technology described herein. In particular, FIG. 9A-9B shows an exemplary implementation of an actor-critic reinforcement learning model, employing deep-deterministic policy gradient techniques. A corresponding pseudocode diagram, showing an exemplary programmatic implementation of a model as described in FIG. 9A-9B, is provided in FIGS. 10A-10C below.

At block 900, the neural networks for the statistical model (e.g., actor network 904, critic network 924, actor target network 926, and critic target network 928) are initialized. The replay buffer R is also initialized at block 900.

At block 902, a reset operation is performed. This may, for example, serve the purpose of clearing episode variables from previous episodes in order to prepare for a new episode. In particular, the reset operation may include resetting a total targeted cell population, total dosages used in the episode, and an initial state s.

At block 904, the state s is provided as input to the actor network. The actor network may produce an output action in the form of dosages of available agents. At block 906, exploration noise may be added to the dosages, such as according to the equation shown in the figure (e.g., Ornstein-Uhlenbeck noise). At block 908, a simulation SIM is performed for time T. This may entail, for example, running a model of cell population evolution for the desired time interval. At block 910, population levels in each bin are updated after time τ has elapsed (e.g., based on the results of the simulation). At block 912, the total dosages used in the episode may be updated, according to the dosages from block 906.

At block 914, based on the updated population levels and total dosages used in the episode, a next state s′ is computed, a reward r is computed, and the done flag is set based on whether the episode has terminated. At block 916, the replay buffer R may be updated to store a tuple representing this transition from one state to another. As shown in the figure, this tuple may contain the state s, the next state s′, the dosages, the reward r, and the done flag. At 918, it is determined whether the size of the replay buffer R exceeds a minimum size. If yes, control moves to the training step process 920.

Training step process 920 may begin at block 922, with randomly sampling a minibatch of transition tuples from replay buffer R updated at block 916. For each transition tuple across the minibatch, the following acts are performed. At block 924, the state s and dosages are provided as input to the critic network, which outputs a predicted q-value (e.g., a predicted action-value function output). At block 926, the next state s′ is provided as input to the actor target network, which outputs next dosages based on the next state. At block 928, the critic target network, based on the next dosages from block 926, as well as the next state from block 922, computes value v_{c} which is used at block 930, in combination with the reward r from block 922, to compute a target q-value. This computation also involves an RL discount factor γ.

At block 932, the predicted q-value and target q-value are used to compute a mean square error loss for the critic, according to the equation shown in the diagram. The resulting MSE loss may be used at block 934 to update the weights of the critic network 924. Then, a policy gradient may be computed at block 936, such that the weights of the actor network 904 may be updated at block 938. At step 940, the weights for the actor target network 926 and critic target network 928 may be soft-updated.

Control then passes away from the training step process 920, and moves to block 942. Note that block 942 may also be reached directly from block 918 in the case where the size of the replay buffer R does not exceed the minimum size. At block 942, the done flag is checked to determine whether the episode has terminated. If yes, control passes to block 902, which performs the reset operation, so as to begin a new episode. If no, then the state s is set to s′ and provided as input to the actor network 904, so as to continue the episode.

FIG. 10A-10C are diagrams showing pseudocode for an example implementation of a training process for training a statistical model for determining a dosage plan, in accordance with some embodiments of the technology described herein. As a note on the index notation used in FIGS. 10A-C, a labels drugs and ranges from 1 to the number of usable available drugs, a labels growth parameters (e.g., birth vs. death rates) and depends on the functional parameterization of the growth rates, and i labels cell populations i=1 to n. As a further note, bracketed and italicized text refer to aspects of a particular implementation example, which may be more specific to the scenario described in the paper titled “Dynamic Control of Stochastic Evolution: A Deep Reinforcement Learning Approach to Adaptively Targeting Emergent Drug Resistance” by Dalit Engelhardt, March 2019 (https://arxiv.org/abs/1903.11373), which is hereby incorporated by reference in its entirety.

FIG. 10A shows the overall training algorithm. The training algorithm includes a number of episodes, from 1 to a maximum number of episodes. Prior to the first episode of FIG. 10A, training preliminaries 1010 may be performed, as shown in FIG. 10B. Within each episode, the training environment is initially reset, then a training loop 1020 is performed, as shown in FIGS. 10C-1 and 10C-2. As should be appreciated from the common language between FIGS. 10A-10C and FIG. 9A-9B, some of the steps of the pseudocode in FIGS. 10A-10C may correspond to blocks illustrated in the flowchart of FIG. 9A-9B.

FIG. 10B shows pseudocode for the training preliminaries 1010. The training preliminaries include establishing parameters, establishing time intervals, and establishing a model of cell population evolution (e.g., a cell population evolutionary dynamics stochastic simulation SIM). It may also include initializing critic and actor networks with respective parameters, initializing critic and actor target networks with respectively identical weights, and initializing a replay buffer.

FIGS. 10C-1 and 10C-2 show pseudocode for the training loop 1020. Some of the steps of pseudocode, specifically in FIG. 10C-2, may correspond to the training step process 920 of FIG. 9A-9B. As shown in FIGS. 10C-1 and 10C-2, the training loop may include performing a number of transitions, and storing the results in the replay buffer. A transition may include selecting dosages based on a previous state using the actor network, then determining a new state, based on the previous state and dosages, using the model of cell population SIM. A reward for the current time step may then be determined based on the previous state, the new state, and the dosages. If the end of the episode has been reached (e.g., the targeted cell populations have been eliminated, the target cell populations have concentrations below a threshold, or the maximal time step for the episode has been reached) then the done flag may be set to true. The transition tuple, including the previous state, new state, reward, dosages, and done flag may then be stored in the replay buffer.

Once the replay buffer reaches a predefined minimum size, a training step may be performed. For each transition in a minibatch sampled from the replay buffer, an MSE loss is computed, the weights of the actor and critic networks are updated, and the weights of the actor and critic target networks are soft-updated. This completes the training step, and the training loop concludes by checking whether the done flag is true: if so, the episode ends (e.g., with a break statement, as shown), and if not, the new state computed in the transition above is set to be the previous state, and the training loop repeats.

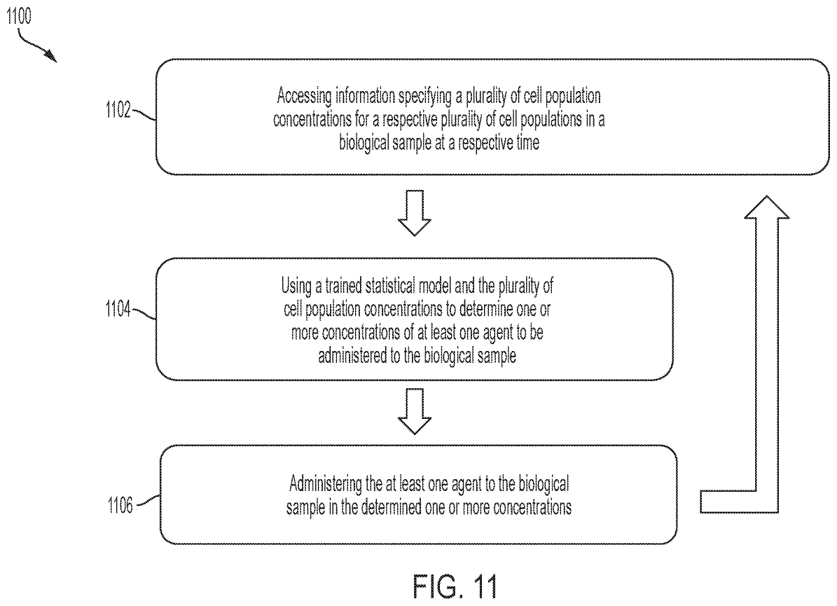

FIG. 11 is a flowchart of an illustrative process 1100 for performing controlled evolution of cells within a biological sample. Process 1100 may take place, for example, in an in vitro setting (e.g., as part an experiment run by an experimenter, for example as described above with respect to FIG. 1A).

At block 1102, information specifying a plurality of cell population concentrations for a respective plurality of cell populations in a biological sample may be accessed. According to some embodiments, the plurality of cell populations may comprise populations of bacteria, such as, for example, E. coli. In an exemplary experiment, it may be the goal of the experimenter to reduce the cell population concentrations for the respective plurality of cell populations below a predefined threshold. The information specifying the plurality of cell population concentrations may, in some examples, be determined experimentally. In some cases, the cell population concentrations may be determined by the experimenter, or they may be determined automatically with lab equipment configured to perform analysis of the biological sample.

At block 1104, a trained statistical model and the plurality of cell population concentrations may be used to determine one or more concentrations of at least one agent to be administered to the biological sample. The statistical model may be developed, for example, according to the techniques described above with respect to FIGS. 7-10, or according to any other suitable techniques. Continuing the example above, the statistical model may be developed by the experimenter, who may establish pre-training preliminaries and develop/train the model according, e.g., to methods illustrated in FIGS. 3 and 9. In some embodiments, the experimenter may simply access a statistical model having previously been developed/trained. Regardless of how the statistical model is obtained, the plurality of cell population concentrations may, according to some embodiments, be provided as input to the trained statistical model, which may determine, as its output, one or more concentrations of at least one agent to be administered to the biological sample.

At block 1106, the at least one therapeutic agent may be administered to the biological sample in the determined one or more concentrations. As described herein including with respect to FIG. 1A, the administration of agents may be performed in a number of ways, which may involve, for example, an experimenter administering the agents, or may involve a mechanism for automatically administering the agents. In some embodiments, after the agents are administered, the method 1100 may return to block 1102. That is, the depicted method for controlled evolution may be a repeated method, involving repetitive steps of accessing cell population concentrations, determining concentrations of agent(s) to be administered, and administering the agent(s) in the determined concentrations. In some embodiments, the method may be carried out by an experimenter in a manual feedback loop. That is, the experimenter may repeatedly obtain cell population concentrations from the lab machinery, supply them to a program running on a computer with the trained statistical model for next-step agent dosages, and then administer the prescribed dosages. The method 1100 may repeat, for example, until the experiment is successful, or until the experiment fails.

FIG. 12A-12B is a chart showing exemplary results of a statistical model trained using the training process of FIG. 8A-8B. In particular, FIG. 12A-12B depicts, in side-by-side charts plotted over time, concentrations of cell populations 1202, 1204, 1206, 1208, and concentrations of a therapeutic agent 1212. In particular, the cell population concentrations are plotted in charts 1200, 1220, 1240, and 1260, while the therapeutic agent concentrations are plotted in charts 1210, 1230, 1250, and 1270. The x-axis for both charts represents time in hours. The y-axis for charts 1210, 1230, 1250, and 1270 represents drug concentrations (measured, in this example, in μg/mL) and the y-axis for charts 1200, 1220, 1240, 1260 represents total targeted cell concentration (measured, in this example, in cells per unit volume). Note that in this example the cell populations 1202, 1204, 1206, and 1208 correspond to the cell populations 502, 504, 506, and 508 depicted in FIG. 5 above, and have corresponding dose-response curves. In the illustrated charts, the concentrations of therapeutic agent 1212 over time are based on the outputs from a statistical model trained using the training process of FIG. 8A-8B. In particular, charts 1210, 1230, 1250, and 1270 represent a treatment policy, developed using the techniques described herein, being applied to the cell environments represented by charts 1200, 1220, 1240, and 1260 respectively. In this example, the therapeutic agent 1212 is applied continuously to the cell environments.

Consider, for example, chart 1200, which shows concentrations of cell populations 1206 and 1202 changing over time. Initially, a high concentration of cell population 1202 is present, while the cell population 1206 is not present at all (i.e., it has a cell population concentration of zero). In chart 1210, the corresponding concentrations of therapeutic agent 1212, which may be applied to the cell environment represented by chart 1200, are depicted. Initially, the concentration of the therapeutic agent 1212 remains substantially stable. However, when cell population 1206 emerges just before hour 20, the corresponding concentration of therapeutic agent 1212 increases, and remains heightened until the cell population 1206 is eliminated just after hour 40. Given the dose-response curves for cell populations 1202 and 1206, as shown in FIG. 5 as 502 and 506 respectively, this policy is very effective: although cell population 1206 has a lower growth rate than cell population 1202 at low therapeutic agent concentrations, cell population 1206 exhibits a much higher degree of resistance to the therapeutic agent than cell population 1202. Thus, in order to efficiently and effectively eliminate cell population 1206, the concentration of the therapeutic agent 1212 increases substantially, as shown in chart 1210, then decreases again once the cell population 1206 has been eliminated.

The other charts depicted in FIG. 12A-12B illustrate similar scenarios involving one or more cell populations of 1202, 1204, 1206, and 1208, and therapeutic agent 1212.

FIGS. 12C, 12D, and 12E illustrate similar scenarios involving two therapeutic agents.

FIG. 13A-13D is a chart showing exemplary training loss and reward of a statistical model trained using the training process of FIG. 8A-8B. In particular, FIG. 13A-13D depicts, in side-by-side charts plotted over number of training episodes, the MSE loss and the end-of-episode reward that may be obtained by the statistical model as it trains over many episodes. FIG. 13A-13D also provides separate charts for a statistical model trained in a one drug scenario, and statistical model trained in a two drug scenario. In particular, charts 1302 and 1304 depict the one drug scenario, while charts 1306 and 1308 depict the two drug scenario.

In chart 1302, the MSE loss for a statistical model being trained over approximately 55,000 episodes is plotted. The corresponding rewards for the same statistical model being trained over the same episodes appear in chart 1304. Although there are some outliers among the data points plotted in charts 1302 and 1304, in general, the MSE loss decreases as the number of episodes increases, while the rewards increase with the number of episodes. Here, this indicates that the model is effectively learning, since the statistical model is intended to minimize loss and maximize reward as part of optimization. A similar trend is apparent in chart 1306, which plots the MSE loss for the two drug scenario, and chart 1308, which plots the corresponding rewards for the two drug scenario. Although a higher degree of variance may be observed in the two drug scenario, as shown, the trend towards decreased loss and increased reward with an increasing number of episodes suggests that the statistical model is effectively learning in the two drug case as well,

FIGS. 12 and 13 show that the techniques described herein provide a significant improvement over conventional techniques for determining dosage plans in cell environments prone to resistance evolution. As described herein, conventional reinforcement learning techniques are not sufficiently adaptable and robust to handle the unexpected feedback that may arise frequently in such cell environments. Conventional techniques also lack the ability to discover, despite system stochasticity and random events, high-preference low-cost policies applicable when such perturbations do not occur. Moreover, they fail to account for the various factors that must be balanced as part of determining dosage plans, such as efficient elimination of cell populations and drug toxicity.

In contrast, the techniques described herein provide significantly improved techniques for combatting the emergence of drug resistance. As shown in FIG. 12A-12B, policies determined according to the techniques described herein are robust in the face of unexpected feedback, such as mutations: for example, charts 1200 and 1210 show how, under such a policy, the concentration of the therapeutic agent 1212 is adaptively increased in order to address the emergence of resistant cell population 1206. Moreover, as further depicted in charts 1200 and 1210, the concentration of therapeutic agent 1212 is adaptively decreased following the elimination of cell population 1206. This reflects the fact that policies determined according to the techniques described herein arise from statistical models trained to account for a wide variety of factors including, in this case, avoiding higher cumulative drug dosages (see, e.g., FIG. 7 and the “Exemplary Statistical Model” section below). As FIG. 13A-13D emphasizes, statistical models trained according to these techniques learn effectively, even in scenarios involving multiple therapeutic agents. Additionally, in FIG. 12A-12B, charts 1220 and 1230 show that models trained according to the techniques described herein are able to produce high-preference low-cost policies when perturbations (e.g., mutations) do not occur.

Aspects of the technology described herein are described further in the following Sections including with reference to non-limiting examples.

### Stochastic Modeling and Simulation of Cell Population Evolution

Described herein is an exemplary implementation of a model of cell population evolution, such as may be used to simulate cell population concentrations over time. In this example, the evolutionary fate of an individual cell during any given time interval depends on the timing of its cell cycle and its interaction with and response to its environment. A model of cell population evolution, according to some embodiments of the techniques described herein, may depend on three processes: cell birth, cell death, and inheritable changes in cell characteristics that lead to observable changes in growth under inhibition by an agent (e.g., a therapeutic agent, such as an antibiotic or other drug). We consider in this example drugs that suppress cell growth by inhibiting processes essential for cell division by binding to relevant targets and thus leading to reduced cell birth rates. In this example, the dose-response relationship can be described by a Hill-type relationship (see, e.g., “The possible effects of the aggregation of the molecules of haemoglobin on its dissociation curves” by Archibald Vivian Hill, 1910, which is hereby incorporated by reference in its entirety; and “Derivation and properties of Michaelis-Menten type and Hill type equations for reference ligands” by Ting-Chao Chou, 1976, which is hereby incorporated by reference in its entirety). Here a modified Hill-type equation may be employed that includes the potential use of multiple drugs with concentrations given by I=(I₁, . . . , Iₘ). The growth rate gᵢ of a particular cell population i as a function of the drug concentrations to which cells are exposed is thus taken to be

$${g}_{i} ( I ) = {β}_{i} ( I ) - {δ}_{i} = \frac{{β}_{i , 0}}{1 + {Σ}_{α} ( {I}_{α} \text{/} {p}_{i , α} )} - {δ}_{i} \qquad ( 1 )$$

where βᵢ is the rate of cell birth, β_{i,0} is its birth rate in a drug-free environment, δᵢ is the rate of cell death, and p_{i,α} describes the extent of resistance of cell population i to drug α. For simplicity, since drugs are assumed here to affect only birth rates, changes that confer resistance are assumed to affect only βᵢ(I) rather than δᵢ. Resources for cell growth are assumed to be limited, with the environmental carrying capacity denoted by K, indicating the total bacterial cell population size beyond which no further population increases take place due to resource saturation.

Evolution is an inherently stochastic process that may depend on processes, as noted above, that individual cells undergo. However, with conventional techniques, agent-based simulations are generally very costly for larger numbers of cells and, moreover, may obscure insight into any approximate deterministic information about the system. At very high population levels (that can be treated as effectively infinite) a deterministic description of system dynamics may be appropriate. In the dynamic growth scenario considered here, large size discrepancies can exist at any time between the various concurrent cell populations, and a single population can decay or proliferate as the simulation progresses. The extent of demographic stochasticity in such systems may thus change in the course of evolution. For efficient modeling at higher population levels, a simulation model whose runtime does not scale with population size may be appropriate, but for accurate modeling a proper estimate of stochasticity may be included. This may be done here by applying methods from stochastic physics to derive a diffusion approximation of a master equation describing the continuous-time evolution of the probability distribution of the population being in some state (n₁, n₂, . . . , n_{d}), where nᵢ are the (discrete) subpopulation levels for the different cell populations. This approximation relies on the assumption that the environmental carrying capacity is large compared to individual subpopulation levels nᵢ and results in the evolution of the system being described by a system of stochastic differential equations (SDEs) governing the time evolution of the cell population levels of the different cell populations given by the continuous variables x=(x₁, . . . , x_{d}), where d is the number of distinct cell populations that may arise. We only consider cell populations whose growth rate g (I) at nonzero drug concentrations is higher than the baseline susceptible cell population (wildtype) due to the negative evolutionary selection experienced by cell populations with lower growth rates. The system evolves according to

$$\frac{{dx}_{i} ( t )}{dt} = ( {β}_{i} ( I ) - {δ}_{i} ) {x}_{i} ( t ) ( 1 - \frac{{\sum }_{j = 1}^{d} {x}_{j} ( t )}{K} ) + \sqrt{( {β}_{i} ( I ) + {δ}_{i} ) {x}_{i} ( t ) ( 1 - \frac{{\sum }_{j = 1}^{d} {x}_{j} ( t )}{K} )} {W}_{i} ( t ) , \qquad ( 2 )$$

Where i=1, . . . , d and the white noise Wᵢ(t) satisfies

$$\{ \begin{matrix}〈 {W}_{i} ( t ) 〉 = 0 \\ 〈 {W}_{i} ( t ) {W}_{i} ( {t}^{′} ) 〉 = δ ( t - {t}^{′} )\end{matrix}$$

From a control standpoint, one advantage of using SDEs over an agent-based model is that it capitalizes on the equations-based description for trajectory estimation and allows that information to be used in feature engineering and reward assignment, as described herein. Note that the stochastic noise in these equations need not be put in ad hoc but may instead emerge naturally from population demographic randomness that arises from demographic processes on the level of single cells. For more realistic dynamics at very low cell levels, when the level of a subpopulation falls below 10 cells, the number of cells is discretized by a random choice (equal probability) to round up or down to the nearest integer cells after each stochastic simulation step; when cell numbers fall below 1 the cell population level is set to zero.

The term decision time step may refer to the length of time between each dosing decision, which may define a reinforcement learning time step, whereas SDE simulation step may refer to a stochastic simulation step. A single decision time step may contain a large number of SDE simulation steps. The evolution of Eq. (2) may be simulated via the Euler-Maruyama method with a step size of 0.01 over the 4-hour decision time step (unless all populations drop to zero before the end of the decision time step is reached).

In this example, mutations are modeled as random events that perturb the system of Eq. 2 by injecting new population members into a particular cell population xᵢ. At the start of each SDE simulation step τₛᵢₘ=0.01, the expected number of baseline-cell type birth events is computed as N_{β}=τₛᵢₘ/β_{bl}x_{bl}, where β_{bl} is the baseline cell birth rate at the current drug concentration and x_{bl} is its population size at the start of the simulation step. N_{β} random numbers rᵢ are then sequentially generated, and where rᵢ≤P_{step,mut}, such that P_{step,mut} is some chosen probability of mutation, a mutation occurs and an increase of 1 cell is randomly assigned with uniform probability to any of the possible (potentially-occurring) non-baseline cell populations. We note that for sufficiently small τₛᵢₘ, where the baseline population does not experience significant changes within a simulation step, this is equivalent to sequentially generating births at the appropriate time intervals and allowing mutations with probability P_{step,mut}. In training, P_{step,mut} was set to 10⁻⁶, in keeping with typical approximate observed rates of occurrence of resistant mutant cells in bacterial populations. Subsequent testing of the policy may be performed on a large range of P_{step,mut} values to ensure policy robustness to uncertainty in the mutation rate.

### Derivation of Eq. 2

Let Aᵢ denote a single cell of phenotype i. We consider d phenotype with equal resource utilization evolving subject to a resource capacity given by K. We initially consider a spatial grid permitting up to one individual of type Aᵢ per site. If empty, individual in site is denoted by E. The rates of birth and death of cells of type i are denoted by \tilde{β}ᵢ and \tilde{δ}ᵢ respectively:
Birth: AᵢE [symbol] AᵢAᵢ
Death: Aᵢ [symbol] E
   At each time step we sample the population. On a fraction η of these events we randomly choose two individuals and allow them to interact, but since we do not consider direct competition effects other than through shared limited resources, we ignore combinations in which both Aᵢ cells were picked and therefore restrict to picking only the combination Aᵢ and E. In a fraction 1−η of these events we choose only one individual randomly (if only E's are drawn, the drawing is done again). The probabilities of picking the different combinations are given by

$$P ( {A}_{i} E ) \text{:} 2 η \frac{{n}_{i}}{K} ( \frac{K - {Σ}_{k} {n}_{k}}{K - 1} )$$

$$P ( {A}_{i} ) \text{:} ( 1 - η ) \frac{{n}_{i}}{K}$$

where nᵢ is the population of phenotype i. We will denote the transition probabilities from state n=(n₁, . . . , n_{d}) and to state n′ by T (n′|n). They are thus given by

$$\{ \begin{matrix}T ( {n}_{1} , … , {n}_{1} + 1 , … , {n}_{d} \vert n ) = 2 η {\tilde{β}}_{i} \frac{{n}_{i}}{K} ( \frac{K - {Σ}_{k} {n}_{k}}{K - 1} ) \approx 2 η {\tilde{β}}_{i} \frac{{n}_{i} ( K - {Σ}_{k} {n}_{k} )}{{K}^{2}} \\ T ( {n}_{1} , … , {n}_{1} - 1 , … , {n}_{d} \vert n ) = ( 1 - η ) {\tilde{δ}}_{i} \frac{{n}_{i}}{K} \end{matrix}$$

where for simplicity we replace K−1→K as we will be considering the large-K limit for the subsequent diffusion approximation. The master equation is given by
(10)

$$\frac{dP ( n , t )}{dt} = {\sum }_{i = 1}^{d} \{ T ( n \vert {n}_{1} , … , {n}_{1} + 1 , . . , {n}_{d} ) P ( {n}_{1} , … , {n}_{1} + 1 , . . , {n}_{d} , t ) + T ( n \vert {n}_{1} , … , {n}_{1} + 1 , . . , {n}_{d} ) P ( {n}_{1} , … , {n}_{1} - 1 , . . , {n}_{d} , t ) - [ T ( {n}_{1} , … , {n}_{1} - 1 , . . , {n}_{d} \vert n ) + T ( {n}_{1} , … , {n}_{1} + 1 , . . , {n}_{d} \vert n ) ] P ( n , t ) \}$$

subject to the initial condition P(n, 0)=δₙ_{₀}_{,n} (Kronecker delta, not to be confused with the death rates δᵢ). We must also impose boundary conditions,

$$\{ \begin{matrix}T ( {n}_{1} , … , {n}_{i} = 0 , … , {n}_{d} \vert {n}_{1} , … , {n}_{i} = - 1 , … , {n}_{d} ) = 0 \\ T ( {Σ}_{i} {n}_{i} = K \vert {Σ}_{i} {n}_{i} = K + 1 ) = 0 \end{matrix}$$

By assuming that the resource capacity K is large compared to the population levels nᵢ, we can approximate φᵢ=nᵢ/K<<1 as continuous variables; we denote P(n₁, . . . , nᵢ±1, . . . , n_{d},t)≡P_{φ}(φᵢ±1) and define

$$\{ \begin{matrix}f ( {φ}_{i} ) ≡ {δ}_{i} {φ}_{i} \\ g ( {φ}_{i} ) ≡ {β}_{i} {φ}_{i} ( 1 - {Σ}_{j}^{d} φ j )\end{matrix}$$

where we have rescaled the death and birth rates as δᵢ ≡(1−η)\tilde{δ}ᵢ and βᵢ≡η\tilde{β}ᵢ. Eqn. (10) can then be rewritten as

$$\frac{{dP}_{φ} ( \vec{φ} , t )}{dt} = {\sum }_{i = 1}^{d} \{ [ f ( {φ}_{i} + \frac{1}{K} ) {P}_{φ} ( {φ}_{1} , … , {φ}_{i} + \frac{1}{K} , … , {φ}_{d} , t ) - f ( {φ}_{i} ) {P}_{φ} ( \vec{φ} , t ) ] + [ g ( {φ}_{i} - \frac{1}{K} ) {P}_{φ} ( {φ}_{1} , … , {φ}_{i} - \frac{1}{K} , … , {φ}_{d} , t ) - g ( {φ}_{i} ) {P}_{φ} ( \vec{φ} , t ) ] \}$$

Taylor expanding around φᵢ to second order we have that

$$h ( {φ}_{i} \pm \frac{1}{K} ) {P}_{φ} ( {φ}_{1} , … , {φ}_{i} \pm \frac{1}{K} , … , {φ}_{d} , t ) = h ( {φ}_{i} ) {P}_{φ} ( \vec{φ} , t ) \pm \frac{1}{K} \frac{\partial ( hP )}{{\partial }_{φ i}} {\vert}_{\vec{φ}} + \frac{1}{2 {K}^{2}} {\sum }_{j} \frac{{\partial }^{2} ( hP )}{\partial {φ}_{j}^{2}} {\vert}_{\vec{φ}}$$

which yields, after rescaling time as τ=t/K, the Fokker-Planck equation

$$\frac{\partial {P}_{φ} ( \vec{φ} , t )}{\partial τ} = {\sum }_{i = 1}^{d} [ \frac{\partial ( ( f - g ) P )}{\partial {φ}_{i}} {\vert}_{\vec{φ}} + \frac{1}{2 K} {\sum }_{j} \frac{{\partial }^{2} ( ( f + g ) P )}{\partial {φ}_{j}^{2}} {\vert}_{\vec{φ}} ]$$

which corresponds to the system of Ito stochastic differential equations

$$d {φ}_{i} = - ( f ( {φ}_{i} ) - g ( {φ}_{i} ) ) dt + \frac{1}{\sqrt{K}} {\sum }_{m} {δ}_{mi} \sqrt{f ( {φ}_{m} ) + g ( {φ}_{m} )} {dW}_{m} ( t )$$

where the white noise Wₖ (t) satisfies

$$\{ \begin{matrix}〈 {W}_{k} ( t ) 〉 = 0 \\ 〈 {W}_{k} ( t ) {W}_{ℓ} ( {t}^{′} ) 〉 = {δ}_{𝓀ℓ} δ ( t - {t}^{′} )\end{matrix} \qquad ( 11 )$$

and δₘᵢ is the Kronecker delta (not to be confused with the death rate δᵢ). We have that

$$\begin{matrix}f ( {φ}_{i} ) \pm g ( {φ}_{i} ) = ( 1 - {\sum }_{j}^{d} {φ}_{j} ) [ {δ}_{i} \frac{{φ}_{i}}{1 - {Σ}_{j}^{d} {φ}_{j}} - {δ}_{i} {φ}_{i} + {δ}_{i} {φ}_{i} \pm {β}_{i} {φ}_{i} ] \\ = {φ}_{i} ( 1 - {\sum }_{j}^{d} {φ}_{j} ) [ {δ}_{i} \frac{{Σ}_{j}^{d} {φ}_{j}}{1 - {Σ}_{j}^{d} {φ}_{j}} + {δ}_{i} \pm {β}_{i} ]\end{matrix}$$

If we redefine xᵢ ≡Kφᵢ as the levels (concentrations) rather than proportions of the populations, we observe that the fraction

$$\frac{{\sum }_{j}^{d} {φ}_{j}}{1 - {\sum }_{j}^{d} {φ}_{j}} = \frac{\frac{1}{K} {\sum }_{j}^{d} {x}_{j}}{1 - \frac{1}{K} {\sum }_{j}^{d} {x}_{j}}$$

approaches 1 in the large-K limit, in which case we have

$$f ( {φ}_{i} ) \pm g ( {φ}_{i} ) \to \frac{1}{K} ( {δ}_{i} \pm {β}_{i} ) {x}_{i} ( 1 - \frac{{\sum }_{j}^{d} {x}_{j}}{K} )$$

yielding the system (2)

$$\frac{d {x}_{i} ( t )}{d t} = ( {β}_{i} - {δ}_{i} ) {x}_{i} ( t ) ( 1 - \frac{{\sum }_{j = 1}^{d} {x}_{j} ( t )}{K} ) + \sqrt{( {β}_{i} + {δ}_{i} ) {x}_{i} ( t ) ( 1 - \frac{{\sum }_{j = 1}^{d} {x}_{j} ( t )}{K} )} {W}_{i} ( t )$$

for i=1, . . . , d. To prevent the term in the square root of the noise from occasionally dipping below zero and resulting in imaginary numbers (due to the cell subpopulations potentially dipping below zero during a simulation step before being set to zero), this term is clipped at zero.

### Exemplary Statistical Model

Described herein is an exemplary implementation of a statistical model for determining dosage plans. In this example, a deep deterministic policy gradient algorithm was employed for training. The exemplary state and action spaces and reward assignment are described below, and the neural network architecture and hyperparameter choices for the neural network and reinforcement learning (RL) components are given.

For this example, in training, the maximal time for an episode was set at 7 days, with dosing decision steps of 4 hours. Although time scales for observations may be technology-dependent and possibly longer, this time interval was put in place in order to demonstrate the ability of the algorithm to handle episodes with many decision time steps, and hence a near-continuous dose adjustment, and produce a robust control policy able to adaptively and responsively adjust to changes in the cell population composition. To put in clinical context the maximal treatment time chosen, we note that one study (Singh et al., Short-course empiric antibiotic therapy for patients with pulmonary infiltrates in the intensive care unit: a proposed solution for indiscriminate antibiotic prescription. American journal of respiratory and critical care medicine, 2000) of an intensive care unit (ICU) patient cohort found that typical ICU antibiotic courses lasted for 4-20 days, with an average length of 10 days; after 7 days of a standard antibiotic course 35% of patients in the study were found to have developed antibiotic resistance (new resistant patterns in the original pathogen) or superinfections (newly-detected pathogenic organisms not originally present). Shorter treatment times (3 days) in that study correlated with a lower rate of antibiotic resistance (15% at 7 days). Stopping antibiotic treatment prematurely, however, runs the risk of failing to eliminate the pathogenic bacteria. Thus, continuous monitoring and proper dose adjustments, as recommended here, are essential in combatting treatment failure in severe infections.

All code (simulation and RL), for this example, was written in Python, with the use of PyTorch for the deep learning components. Though any other suitable software framework may be used, as aspects of the technology described herein are not limited in this respect.

### Exemplary State Space

In this example, at each decision time step an observation of the current composition of the cell population, i.e. types and respective concentrations x=(x₁, . . . , x_{d}) is made. Cell types that are known to potentially arise but are presently unobserved are assigned xᵢ=0. As a result of model knowledge in the form of the SDE system (e.g., Eq. 2), however, additional feature combinations of x can be used to supplement and improve learning. In particular, the current growth rate of the total cell population is highly indicative of drug effectiveness, but it is typically difficult to measure and necessitates taking multiple observations over some fixed time interval (e.g. 1 hour) prior to the end of a decision time step. Instead, the instantaneous growth rate can be calculated as

$${g}_{a l l} ( t ) = \frac{{\sum }_{i} {\dot{x}}_{i} ( t )}{\sum {x}_{i} ( t )} \qquad ( 3 )$$

and approximated directly from observed features, xₜ, at time t by noting that in the deterministic limit the numerator is simply given by

$${\sum }_{i} {\dot{x}}_{ι} ( t ) = ( 1 - \frac{{\sum }_{i} {x}_{i}}{K} ) \times {\sum }_{i} {g}_{i} ( I ) {x}_{i} ( t ) \qquad ( 4 )$$

where the gᵢ are fixed and known (Eqn. 1) during any decision time step. Hence gₐₗₗ is a function exclusively of the action taken (doses chosen) and observations of cell concentrations xₜ.

Use of this feature combination, which would not be known in a blackbox simulation, may be instrumental in shaping the reward signal at non-terminal time steps and in leading to optimal policy learning, suggesting that such incorporation of information could in general assist in driving policy convergence when model knowledge is available but a model-free approach is preferred due to the complexity of the dynamics.

### Exemplary Action Space

In this example, training was performed with both a single drug Iₜ and two drugs (Iₜ^{α},Iₜ^{β}). The effect of the drugs on the cell populations is expressed through the dose-response as in Eq. (1) and the SDE system of Eq. (2). Drugs were set to take a continuous range of values from zero up to some maximal value Iₘₐₓ and this maximal value was used for reward scaling rather than for placing a hard limit on the amount of drug allowed to be administered. It was set for both drugs at 8 times the highest ρ value in the set {ρ_{i,α}} for all drugs α. This very high value was chosen in order to allow enough initial dosing climb during training; in practice, conservative dosing was implemented through the reward assignment alone.

### Exemplary Reward Assignment

In this example, while the state space includes information on separate cell subpopulations, the terminal (end-of-episode) reward is assigned based on only the total cell population. As noted above, a successful episode concludes in the elimination of the targeted cell population by the maximal allowed treatment time Tₘₐₓ. In training, different drugs are assigned preference through penalty weights w_{α}, so that use of a last-line drug will incur a higher penalty than that of a first-line drug, α=1. To penalize for higher cumulative drug dosages, the reward at the end of a successful episode is assigned as

$${r}_{success} = {c}_{end . success} ( 1 - \frac{{\sum }_{α}^{m} ( \frac{{w}_{α}}{{w}_{1}} ) {\sum }_{n = 1}^{{T}_{max / T}} {I}_{α , n}}{{c}_{1} {I}_{max} {T}_{max} / τ} ) \qquad ( 5 )$$

where I_{α,n} is the dosage of drug α administered at the nth decision time step, c₁, c_{end,success}>0 are constants, and τ is the length of each decision time step. If an episode fails (the cell population is above zero by Tₘₐₓ) a negative reward is assigned with an additional penalty that increases with a higher ratio of remaining cell concentrations to the initial cell population in order to guide learning:

$${r}_{f a i l u r e} = - {c}_{end , fail} [ 1 + log ( 1 + \frac{{\sum }_{i} {x}_{i} ( {T}_{max} )}{{\sum }_{i} {x}_{i} ( 0 )} ) ] \qquad ( 6 )$$

where c_{end,fall}>0. Since the episodes can be long, in order to address the credit assignment problem (Minsky, Steps toward artificial intelligence. Proceedings of the IRE, 1961) a guiding signal was added at each time step in the form of potential-based reward shaping (Ng et al., Policy invariance under reward transformations: Theory and application to reward shaping. In ICML, volume 99, 1999). Policy optimality was proved to be invariant under this
type of reward signal, and it can thus provide a crucial learning signal in episodic tasks. It is given by

rₛₕₐₚₑ(sₜ,a,s_{t+τ})=γΦ(s_{t+τ})−Φ(sₜ) (7)

where γ is the RL discount factor, Φ: S→ [symbol] is the potential function, and S is the state space. Since the drugs are assumed here to affect cell mechanisms responsible for cell birth, population growth provides a direct indicator of the efficacy of the drug. The potential function was therefore set here to

$$Φ = - {c}_{Φ} \frac{{g}_{all}}{{g}_{max}}$$

where c_{Φ}>0, gₐₗₗ is given by Eq. 3, and gₘₐₓ is the zero-drug full-population deterministic growth rate at t=0 (see also Eq. 4):

$${g}_{max} = ( 1 - \frac{{\sum }_{i} {x}_{i} ( t = 0 )}{K} ) \frac{{\sum }_{i}^{d} {x}_{i} ( t = 0 ) {g}_{i} ( {I}_{α} = 0 , ∀ α )}{{\sum }_{i}^{d} {x}_{i} ( t = 0 )} \qquad ( 8 )$$

Although incentives for low dosing were built into the terminal reward (Eq. 5), for guiding training toward low dosing, some incentives were provided for low dosing at each decision time step. The step reward was therefore assigned as the sum of the shaping reward and a dosage penalty:

$${r}_{s t e p} = {r}_{s h a p e} - Θ ( {\sum }_{i} {x}_{i} ) {\sum }_{a}^{m} {{w}_{α} ( {I}_{α} / {I}_{max} )}^{2} \qquad ( 9 )$$

where η_{α} is the specific penalty for each drug, and Θ(Σᵢxᵢ) is a binary-valued function explained below.

An issue peculiar to the problem of a population growing under limited resources is that if insufficient dosing is applied and the population subsequently quickly grows and reaches its carrying capacity, very little change in growth (or population) will occur yet the state of infection will be at its maximal worst state. However, the shaping reward (7), which is based on changes from one decision time step to the next, will provide insufficient penalty for this (with the penalty arising solely from the γ weighing). On the other hand, as a result of the inhibitor penalty in (9), the algorithm may continue to reduce the dosing even further rather than increasing it to escape this suboptimal policy. The result is that the targeted cell population proliferates at maximal capacity while dosing fluctuates around the minimal value of zero drug administration. This zero-dosage convergence problem was successfully resolved by assigning a significantly lower (20-fold) weight to the mid-episode dosage penalty if the total cell population exceeded the initial cell population by more than 1 cell for every initially present 10⁴ cells. The incorporation of the binary penalty function led to significantly improved stability and reduced sensitivity to the exact choice of w_{α}.

### Exemplary Neural Network Architecture and Hyperparameter Choices

In this example, the actor and critic network (and respective target network) architecture used is the same as base DDPG with the notable difference that significant performance improvement was obtained with the addition of an extra hidden layer (30 units for single-drug training, 45 units for dual-drug training) in the actor network and that narrower hidden layers were implemented in both single-drug (40 and 30 units, respectively) and dual-drug (60 and 45 units, respectively) training to avoid overfitting. All hidden layers employed rectified linear activation functions; the output layer of the actor was set to a hard tanh with range between 0 and a chosen maximum dose in order to allow for the dosage to drop to exactly zero (particularly important when multiple drugs are considered). In this example, weights were initialized randomly but biases were set to a positive value informed by the evolution of the deterministic part of (2) (see below for a description). This helped prevent training from becoming trapped early on in a suboptimal policy in which the minimal control (0) was applied at every time step.

No batch normalization was used at hidden layers, but features were rescaled prior to being added to the replay buffer in the following manner. Cell concentrations xᵢ were rescaled as

$${x}_{i} \to \frac{log ( {x}_{i} + 1 )}{log ( K )}$$

Since cell concentrations can vary by multiple orders of magnitude, this was found to be helpful for smooth convergence. The growth rate gₐₗₗ was rescaled as

$${g}_{a l l} \to \frac{{g}_{a l l} - {g}_{min}}{{g}_{max} - {g}_{min}}$$

Where gₘₐₓ was defined in (8) and gₘᵢₙ was set to the negative of the death rate, −δ, which may be the lowest growth rate (δ is the highest rate of decline) possible in the system.

In this example, Adam optimization was used with learning rates of 10⁻⁵ for the actor network and 10⁻⁴ for the critic network, as learning was found to be unstable for higher rates. Training was done with mini-batch size of 128 and a replay buffer of size 10⁶. Soft updates to the target networks were done with τ=0.001 and the RL discount factor was set at γ=0.99. Exploration was done with an Ornstein-Uhlenbeck process with θ=0.15, σ=0.3, and δₜ=10⁻². Parameters used in the reward choice that resulted in successful training were c_{end,success}=40, w₁=w₂=1, c₁=2, c_{end,fail}=20, c_{Φ}=10. Single-drug training was done with both η₁=16 and η₁=4. Combination therapy (two drugs) training was done with η₂=3×η₁ under the assumption that the drug able to effectively target the higher-resistance cells is a last-resort drug that also involves higher toxicity.

### Exemplary Bias Initialization

In this example, if we assume the lowest-variance policy (constant uniform dosing) and permit no mutations to occur, then in deterministic evolution the number of baseline-phenotype cells under constant dose I starting from a population x₀ will be given at time t by the solution of the noise-free d=1 system

equivalent of (2):

$$x ( t ) = \frac{{x}_{0} {e}^{g ( I ) t}}{1 + \frac{{x}_{0}}{K} [ {e}^{g ( I ) t} - 1 ]} \qquad ( 12 )$$

### Where

$$g ( I ) = \frac{β}{1 + \frac{I}{ρ}} - δ . \qquad$$

If the population must be reduced to xₘᵢₙ (here, <1 cell) within a time Tₘₐₓ, then the dosage must be no less than

$${I}_{min} = ρ [ \frac{β {T}_{max}}{δ {T}_{max} - log ( \frac{{x}_{0} ( K - {x}_{min} )}{{x}_{min} ( K - {x}_{0} )} )} ]$$

for the treatment to be successful. Initial cell populations were allowed to vary by up to an order of magnitude; by taking the maximum value allowed in this range we compute Iₘᵢₙ. and set the bias to five times this value or to 75% of the maximal inhibitor value, whichever is smallest (typically 5Iₘᵢₙ). xₘᵢₙ was set to just under 1 cell (0.98) as the population falling below a single cell marked the conclusion of a successful episode. Initializations for both actor and critic networks were saved and reused in subsequent experiments.

### Computer Implementation

An illustrative implementation of a computer system 1400 that may be used in connection with any of the embodiments of the disclosure provided herein is shown in FIG. 14. The computer system 1400 may include one or more processors 1410 and one or more articles of manufacture that comprise non-transitory computer-readable storage media (e.g., memory 1420 and one or more non-volatile storage media 1430). The processor(s) 1410 may control writing data to and reading data from the memory 1420 and the non-volatile storage device 1430 in any suitable manner, as the aspects of the technology described herein are not limited in this respect. To perform any of the functionality described herein, the processor(s) 1410 may execute one or more processor-executable instructions stored in one or more non-transitory computer-readable storage media (e.g., the memory 1420), which may serve as non-transitory computer-readable storage media storing processor-executable instructions for execution by the processor(s) 1410.

The terms “program” or “software” are used herein in a generic sense to refer to any type of computer code or set of processor-executable instructions that can be employed to program a computer or other processor to implement various aspects of embodiments as described herein. Additionally, in some embodiments, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided including with reference to FIGS. 2-3, 8-9, and 11. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

What is claimed is:

1. A computer-implemented method for determining a dosage plan for administering at least one therapeutic agent to a subject, the method comprising:
using at least one computer hardware processor to perform:
accessing information specifying a plurality of cell population concentrations for a respective plurality of cell populations in a biological sample from a subject; and
determining the dosage plan using a trained statistical model and the plurality of cell population concentrations, the dosage plan including one or more concentrations of the at least one therapeutic agent to be administered to the subject at one or more respective different times.

2. The method of claim 1, wherein determining the dosage plan comprises:
determining the one or more concentrations of the at least one therapeutic agent; and
determining the one or more respective different times for administering the one or more concentrations of the at least one therapeutic agent.

3. The method of claim 1, wherein determining the dosage plan comprises:
accessing information specifying the one or more respective different times; and
determining the one or more concentrations of the at least one therapeutic agent for the one or more respective different times.

4. The method of claim 1 or any other preceding claim, wherein determining the dosage plan comprises:
providing the plurality of population concentrations as input to the trained statistical model; and
determining the dosage plan using the output of the trained statistical model.

5. The method of claim 1 or any other preceding claim, wherein each one of the plurality of cell populations has respective dose-response characteristics different from dose-response characteristics of other ones of the plurality of cell populations.

6. The method of claim 1 or any other preceding claim,
wherein the at least one therapeutic agent includes a first therapeutic agent,
wherein the plurality of cell populations includes a first cell population associated with first dose-response characteristics for the first therapeutic agent and a second cell population associated with second dose-response characteristics for the first therapeutic agent, and
wherein the first dose-response characteristics are different from the second dose-response characteristics.

7. The method of claim 6 or any other preceding claim, wherein a measure of difference between the first dose-response characteristics and the second dose-response characteristics is above a threshold.

8. The method of claim 6 or any other preceding claim, wherein the first dose-response characteristics comprise first parameters for a first dose-response curve and the second dose response characteristics comprise second parameters for a second dose-response curve, wherein the first parameters are different from the second parameters.

9. The method of claim 1 or any other preceding claim, wherein the trained statistical model includes a neural network model.

10. The method of claim 9, wherein the neural network model includes a deep neural network model.

11. The method of claim 10, wherein the deep neural network model includes one or more convolutional layers.

12. The method of claim 10, wherein the deep neural network model includes one or more fully connected layers.

13. The method of claim 1 or any other preceding claim, wherein the trained statistical model is trained using a reinforcement learning technique.

14. The method of claim 13 or any other preceding claim, wherein the trained statistical model is trained using an actor-critic reinforcement learning technique.

15. The method of claim 14 or any other preceding claim, wherein the trained statistical model includes a trained actor network trained using an actor-critic reinforcement learning algorithm.

16. The method of claim 15 or any other preceding claim, wherein the trained statistical model is trained using a deep deterministic policy gradient algorithm.

17. The method of claim 16 or any other preceding claim, wherein the trained statistical model is trained using training data generated using at least one model of cell population evolution.

18. The method of claim 17 or any other preceding claim, wherein the training data is generated using stochastic simulations of the at least one model of cell population evolution.

19. The method of claim 17 or any other preceding claim, wherein the at least one model of cell population evolution includes a set of differential equations representing a time evolution of the plurality of cell population concentrations for the respective plurality of cell populations.

20. The method of claim 19 or any other preceding claim, wherein the at least one therapeutic agent includes a first therapeutic agent, and wherein the set of differential equations depends on dose-response characteristics for the first therapeutic agent.

21. The method of claim 19 or any other preceding claim, wherein the at least one model of cell population evolution models concentration changes among cell populations in the plurality of cell populations.

22. The method of claim 19 or any other preceding claim, wherein the at least one model of cell population evolution models birth of a new cell population.

23. The method of claim 19, wherein the at least one model of cell population evolution models death of a cell population in the plurality of cell populations.

24. The method of claim 1, wherein the at least one therapeutic agent is a small molecule, a protein, a nucleic acid, gene therapy, a drug approved by regulatory approval agency, a biological product approved by a regulatory approval agency, or some combination thereof.

25. The method of claim 1, further comprising administering the at least one therapeutic agent to the subject in accordance with the dosage plan.

26. At least one non-transitory computer-readable storage medium storing processor-executable instructions that, when executed by a computer hardware processor, cause the computer hardware processor to perform a method for determining a dosage plan for administering at least one therapeutic agent to a subject, the method comprising:
accessing information specifying a plurality of cell population concentrations for a respective plurality of cell populations in a cell environment from a subject; and
determining the dosage plan using a trained statistical model and the plurality of cell population concentrations, the dosage plan including one or more concentrations of the at least one therapeutic agent to be administered to the subject at one or more respective different times.

27. A system, comprising:
at least one computer hardware processor; and
at least one non-transitory computer-readable storage medium storing processor-executable instructions that, when executed by the computer hardware processor, cause the computer hardware processor to perform a method for determining a dosage plan for administering at least one therapeutic agent to a subject, the method comprising:
accessing information specifying a plurality of cell population concentrations for a respective plurality of cell populations in a cell environment from a subject; and
determining the dosage plan using a trained statistical model and the plurality of cell population concentrations, the dosage plan including one or more concentrations of the at least one therapeutic agent to be administered to the subject at one or more respective different times.

28. A computer-implemented method for training a statistical model to determine a dosage plan for administering at least one therapeutic agent to a subject, the method comprising:
using at least one computer hardware processor to perform:
generating training data for training the statistical model at least in part by using information specifying an initial plurality of cell population concentrations for a respective plurality of cell populations, and at least one model of cell population evolution;
training the statistical model using the training data to obtain a trained statistical model; and
storing the trained statistical model.

29. The method of claim 28, further comprising:
accessing information specifying, for the subject, a plurality of cell population concentrations for the respective plurality of cell populations; and
determining, using the trained statistical model and the plurality of cell population concentrations, the dosage plan for administering the at least one therapeutic agent to the subject, wherein the dosage plan includes one or more concentrations of the at least one therapeutic agent to be administered to the subject at one or more respective different times.

30. The method of claim 28 or any other preceding claim, wherein each one of the plurality of cell populations has respective dose response characteristics different from dose-response characteristics of other ones of the plurality of cell populations.

31. The method of claim 28 or any other preceding claim, wherein training the statistical model comprises using a reinforcement learning technique.

32. The method of claim 28 or any other preceding claim, wherein training the statistical model comprises using an actor-critic reinforcement learning technique.

33. The method of claim 32 or any other preceding claim, wherein training the statistical model comprises using a deep deterministic policy gradient algorithm.

34. The method of claim 28 or any other preceding claim, wherein the training data is generated using stochastic simulations of the at least one model of cell population evolution.

35. The method of claim 34 or any other preceding claim, wherein the at least one model of cell population evolution includes a set of differential equations representing a time evolution of the initial plurality of cell population concentrations for the respective plurality of cell populations.

36. The method of claim 35 or any other preceding claim, wherein the at least one therapeutic agent includes a first therapeutic agent, and wherein the set of differential equations depends on dose-response characteristics for the first therapeutic agent.

37. At least one non-transitory computer-readable storage medium storing processor-executable instructions that, when executed by a computer hardware processor, cause the computer hardware processor to perform a method for training a statistical model to determine a dosage plan for administering at least one therapeutic agent to a subject, the method comprising:
generating training data for training the statistical model at least in part by using information specifying an initial plurality of cell population concentrations for a respective plurality of cell populations, and at least one model of cell population evolution;
training the statistical model using the training data to obtain a trained statistical model; and
storing the trained statistical model.

38. A system, comprising:
at least one computer hardware processor; and
at least one non-transitory computer-readable storage medium storing processor-executable instructions that, when executed by the computer hardware processor, cause the computer hardware processor to perform a method for training a statistical model to determine a dosage plan for administering at least one therapeutic agent to a subject, the method comprising:
generating training data for training the statistical model at least in part by using information specifying an initial plurality of cell population concentrations for a respective plurality of cell populations, and at least one model of cell population evolution;
training the statistical model using the training data to obtain a trained statistical model; and
storing the trained statistical model.

39. A method for performing controlled evolution of cells within a cell environment, the method comprising:
(A) accessing information specifying a plurality of cell population concentrations for a respective plurality of cell populations in the cell environment;
(B) using a trained statistical model and the plurality of cell population concentrations to determine one or more concentrations of at least one agent to be administered to the cell environment; and
(C) administering the at least one agent to the cell environment in the determined one or more concentrations.

40. The method of claim 39, wherein the acts (A), (B), and (C) are performed repeatedly.

41. The method of claim 40 or any other preceding claim, wherein the method is performed by automated lab machinery comprising at least one computer hardware processor.

42. The method of claim 41 or any other preceding claim, wherein the trained statistical model is trained using training data generated using at least one model of cell population evolution.

43. The method of claim 41 or any other preceding claim, wherein the training data is generated using stochastic simulations of the at least one model of cell population evolution.

44. The method of claim 43 or any other preceding claim, wherein the at least one model of cell population evolution includes a set of differential equations representing a time evolution of the plurality of cell population concentrations for the respective plurality of cell populations.

45. The method of claim 44 or any other preceding claim, wherein the at least one agent includes a first agent and the set of differential equations depends on dose-response characteristics for the first agent.

46. The method of claim 45 or any other preceding claim, wherein the trained statistical model includes a neural network model.

47. At least one non-transitory computer-readable storage medium storing processor-executable instructions that, when executed by a computer hardware processor, cause the computer hardware processor to perform a method for performing controlled evolution of cells within a cell environment, the method comprising:
(A) accessing information specifying a plurality of cell population concentrations for a respective plurality of cell populations in the cell environment;
(B) using a trained statistical model and the plurality of cell population concentrations to determine one or more concentrations of at least one agent to be administered to the cell environment; and
(C) administering the at least one agent to the cell environment in the determined one or more concentrations.

48. A system, comprising:
at least one computer hardware processor; and
at least one non-transitory computer-readable storage medium storing processor-executable instructions that, when executed by the computer hardware processor, cause the computer hardware processor to perform a method for performing controlled evolution of cells within a cell environment, the method comprising:
(A) accessing information specifying a plurality of cell population concentrations for a respective plurality of cell populations in the cell environment;
(B) using a trained statistical model and the plurality of cell population concentrations to determine one or more concentrations of at least one agent to be administered to the cell environment; and
(C) administering the at least one agent to the cell environment in the determined one or more concentrations.
